(19) United States
(12) Patent Application Publication
(10) Pub. No.: US 2006/0034767 A1
(43) Pub. Date: Feb. 16, 2006

(54) Targeting and tracking of cells to specific organs and tissues in vivo

(75) Inventors: Lawrence G Lum, Coventry, RI (US); Randall J Lee, Hillsborough, CA (US); Nicola Mitri Kouttab, Jamestown, RI (US)

Correspondence Address: EDWARDS & ANGELL, LLP, P.O. BOX 55874, BOSTON, MA, 02205, US

(73) Assignee: Roger Williams Medical Center

(21) Appl. No.: 10/520,431

(22) PCT Filed: Jul. 3, 2003
(86) PCT No.: PCT/US03/21142

§ 371 Date: Jul. 27, 2005

Related U.S. Application Data
(60) Provisional application No. 60/393,961, filed on Jul. 5, 2002.

(51) Int. Cl. A61K 49/00; A61K 39/395

(52) U.S. Cl. 424/9.6; 424/133.1

(57) ABSTRACT

The present invention provides a method for tracking and targeting cells in viva. In particular, cells are isolated purified and armed with bispecific antibodies which are directly labeled with a detectable marker, or a second antibody specific for a region of the bispecific antibody is labeled with a detectable marker. The bispecific antibody is specific for a surface antigen of the cell and the second specificity of the specific antigen is for an antigen expressed on cells of tissues, organs or tumors. The armed and labeled cells can be tracked starting from the point of introduction into the animal until the cell reaches the target of interest by phenotyping cells from patient samples, obtained at different time intervals and locations, post-infusion. The isolated and purified cells are useful in any functional assay.

### BACKGROUND OF THE INVENTION

1. Field of the Invention

The present invention provides a method for tracking and targeting cells in vivo. In particular, cells are armed with bispecific antibodies which are directly labeled with a detectable marker, or a second antibody specific for a region of the bispecific antibody is labeled with a detectable marker. The bispecific antibody is specific for a surface antigen of the cell and the second specificity of the bispecific antigen is for an antigen expressed on cells of tissues, organs or tumors. The armed and labeled cells can be tracked starting from the point of introduction into the animal until the cell reaches the target of interest by phenotyping cells from patient samples, obtained at different time intervals and locations, post-infusion.

2. Background

Multicellular animals are derived from a clone of cells descended from a single original cell, the fertilized egg. Embryogenesis involves the division and differentiation of multipotential cells, each cell having the ability to develop into multiple cellular lineages. Phenotypically, the cells of such lineages can vary substantially, such as blood cells, muscle cells and neural cells, being specialized.

A wide spectrum of diseases may be treated based upon both the possession of a population of cells having multi-lineage potential and an understanding of the mechanisms that regulate embryonic cell development. For example, the capacity to generate a new population of hematopoietic cells is the basis of bone marrow transplantation, which is currently used as a treatment for a growing number of diseases including anemia, leukemia and breast cancer. In addition, transplantation of genetically altered multipotential cells has been considered as potential therapy for a variety of different diseases including AIDS.

Mammalian hematopoietic (blood) cells provide a diverse range of physiologic activities. Hematopoietic cells are divided into lymphoid, myeloid and erythroid lineages. The lymphoid lineage, comprising B, T and natural killer (NK) cells, provides for the production of antibodies, regulation of the cellular immune system, detection of foreign agents in the blood, detection of cells foreign to the host, and the like. The myeloid lineage, which includes monocytes, granulocytes, megakaryocytes, as well as other cells, monitors for the presence of foreign bodies, provides protection against neoplastic cells, scavenges foreign materials, produces platelets, and the like. The erythroid lineage provides the red blood cells, which act as oxygen carriers.

Despite the diversity of the nature, morphology, characteristics and function of hematopoietic cells, it is presently believed that these cells are derived from a single cell population, termed hematopoietic “stem cells.” Unlike more “mature” blood cells, stem cells are capable of self-regeneration but may also divide into progenitor cells that are no longer pluripotent and have a limited self-regeneration. These progenitor cells divide repeatedly to form more mature cells which eventually become terminally differentiated to form the various mature hematopoietic cells. Thus the large number of mature hematopoietic cells is derived from a small reservoir of stem cells by a process of proliferation and differentiation.

Progenitor cells mature into bipotential cells and then become lineage committed, that is, are incapable of maturing into more than one lineage. The use of the words progenitor or progenitor cells indicates cell populations which are no longer stem cells but which have not yet become terminally differentiated. The use of the word lymphoid, myeloid or erythroid in conjunction with progenitor indicates the potential cell populations into which the progenitor is capable of maturing.

Highly purified populations of stem cells currently find use in repopulation of the entire hematopoietic system. Purified progenitor cells of individual lineages would find use in repopulating or augmenting the various lineages. Although use of autologous cells would be highly beneficial in therapeutic applications, there is a need in the art to target cells to a desired location in the patient in need of such therapy and more importantly to be able to track the location and identify the cells used in such applications.

### SUMMARY OF THE INVENTION

The present invention provides a method for tracking and targeting cells to organs, and tissues in vivo. The cells are tracked starting from the point of introduction into the animal until the stem cell reaches the target of interest. In particular, the invention provides for the phenotypic identification of the infused cells at any time point or location post-infusion. A particular advantage is using stem cells, which are assimilated by the target tissue or organ of interest and, due to the in vivo target microenvironment, develop into the cell type of the desired target. The therapeutic advantages are many and include the repair of tissues caused by a variety of factors, organs, cell linings, replacement of necrotic tissues and the like. The method is important especially in the areas of chemotherapy, cancers, autoimmune diseases, rejuvenation of necrotic tissues and organs. Especially of interest is the use of the method to repair organs such that organ transplantation may be overcome.

In particular, the invention provides methods for tracking cells in vivo. In a preferred embodiment, the method for tracking stem cells comprises the steps of:
(a) isolating and purifying stem cells from a subject:
(b) providing a chemically heteroconjugated bispecific antibody with a binding site specific for a stem cell antigen and a binding site specific for a target antigen in a patient; and,
(c) arming the stem cells with the bispecific antibody under conditions wherein;
   (i) the bispecific antibody binds to the stem cells via the c-kit ligand; and,
   (ii) the second antigenic binding site of the bispecific antibody is free to bind to the target antigen; and,
   (iii) binding of a labeled antibody to the Fc region of the bispecific antibody; or,
   (iv) fluorescently labeling the bispecific antibody thereby a secondary labeled antibody is not required; and,
(d) reinfusing the armed and labeled stem cells into a patient; and,
(e) tracking the armed and labeled stem cells by extracting samples from the patient at different time intervals; and,
(f) identifying the armed and labeled cells by phenotyping the cells using flow cytometry cell-sorting.
(g) identifying the armed and labeled cells by immunohistochemical staining or other methods to detect the primary antibody on the cells in various target tissues such as bone marrow, spleen, liver, pancreas, lungs, neural tissue, gastrointestinal track, heart, vascular endothelium, etc.

In particular, the use of characteristic phenotypic markers which identify the cell population can be used to track the armed and labeled cells. Examples include, but not limited to: CD2, CD3, CD8, CD10, CD19, CD20, CD14, CD15, CD16, CD33, CD34, CD38, HLA-DR, C-Kit, Thy, Rho, CD45RA and the like, in animals, such as human, mice, primates and the like. The bispecific antibodies of the invention can recognize any antigenic determinant, desired by the user.

In one aspect of the invention, the bispecific antibody is specific for c-kit ligand of stem cells and myocardial antigens. The specificity of the bispecific antibody for myocardial antigens, such as, for example, VCAM-1, NCAM-1, PECAM etc., targets the stem cell to the heart.

In a preferred embodiment, a secondary antibody which is fluorescently labeled and specific for a region of the bispecific antibody, such as the Fc portion. In another preferred embodiment, the bispecific antibody is labeled directly thereby, by-passing the need for a second antibody.

In one aspect of the invention, the armed and labeled stem cells home to, and bind to the target tissue antigens. The stem cells accumulate at the target antigen site and differentiate into cells typical of the targeted tissue or organ. The location of the cells is verified by taking a patient sample, such as a blood sample or a biopsy of the targeted tissue or organ. The sample is subjected to cell sorting assays such as flow cytometry. Preferably, the samples are taken at different time intervals after reinfusion of the stem cells to track the location of the armed and labeled cells and to evaluate their functional capacity of the stem cells in stem cell assays or retransplantation assays. The numbers of armed and labeled cells at a particular time interval and/or in vivo location are quantitatively assessed by comparing the number of armed and labeled cells that were reinfused with the number of armed and labeled cells present in a sample at the particular time interval and/or in vivo location by flow cytometry. In accordance with the invention, the blood samples and target tissue samples taken from a patient at a particular time interval and quantitatively assessed using flow cytometry, is indicative of in vivo homing progress of armed and labeled stem cells to target tissues. In accordance with the invention, the blood samples and target tissue samples taken from a patient at a particular time interval will be sorted and tested for putative specific functions. The sorted cells can be used in any functional assay, depending on the cellular population. For example, cytokine assays, ELISA's, B cell assays, T-cell assays such as cytotoxic assays, proliferation assays, and the like.

In another preferred embodiment, the invention provides a method for treating a patient suffering from cancer, comprising the steps of:
(a) isolating peripheral blood mononuclear cells from a patient suffering from cancer;
(b) activating of T cells by ex vivo stimulation with soluble anti-CD3 monoclonal antibody;
(c) arming of activated T cells with bispecific antibodies capable of binding to the T cell receptor complex of a T cell, and to tumor-associated antigens on a tumor cell, under conditions wherein;
   (i) bispecific antibody binds to said T cells, tumor cells, and Fc-receptor positive cells,
   (ii) activation of said T cells by said antibody binding to the tumor target,
   (iii) redirection of said T cells and Fc-receptor positive cells to said tumor cells,
   (iv) destruction of said tumor cells by said activated and armed T cells; and,
(d) binding of a labeled secondary antibody specific for the Fc region of the bispecific antibody; or,
(e) directly labeling the bispecific anti-body with a detectable marker; and,
(f) reinfusing the armed and labeled unactivated and/or activated T cells into a patient.

In one aspect of the invention, the bispecific antibody is comprised of two monoclonal antibodies. However, the bispecific antibody can be comprised of two polyclonal antibodies or an engineered bispecific antibody. Preferably, each of the specificities of the bispecific antibody are directed to a tumor antigen and the T cell receptor complex. Antibodies can be raised against any tumor antigen from a patient. Thus the targeting of the T cell can be individually tailored as the tumor displays different antigens. For tracking purposes, the bispecific antibody can be directly labeled or a second antibody specific for a region of the bispecific antibody is labeled. Detection of the armed cell is preferably through cell sorting techniques such as flow cytometry. For example, wherein samples are taken at different time intervals after reinfusion of the T cells to track the location of the armed and labeled T cells. The numbers of armed and labeled cells at a particular time interval and/or in vivo location are quantitatively assessed by comparing the number of armed and labeled cells that were reinfused with the number of aimed and labeled cells present in a sample at the particular time interval and/or in vivo location by flow cytometry. As mentioned above, the isolated and purified T cells can also be used in T cell assays, cyokine assays, and any functional assay, for conducting further analyses such as maturity, antigen specificity and the like.

In general, the invention provides for a method for tracking cells in vivo at any desired location, the method comprising
isolating and purifying cells from a subject; and,
providing a chemically heteroconjugated bispecific antibody with a binding site specific for cellular antigen and a binding site specific for a target antigen in any location in a patient, and,
arming the isolated cells with the bispecific antibody under conditions wherein;
   (i) the bispecific antibody binds to a specific antigen on the isolated cell; and,
   (ii) the second antigenic binding site of the bispecific antibody is free to bind to a target antigen; and,
   (iii) binding of a labeled antibody to the Fc region of the bispecific antibody; or,
   (iv) fluorescently labeling the bispecific antibody thereby a secondary labeled antibody is not required; and,
(d) reinfusing the armed and labeled cells into a patient; and,
(e) tracking the armed and labeled cells by extracting samples from the patient at different time intervals; and,
(f) identifying the armed and labeled cells using flow cytometry cell sorting.

In one aspect, the cells used are from any source, such as bone marrow cells, stem cells, hematopoietic stem cells, erythroid stem cells and cells of the immune system. Examples include, lymphocytes, NK cells, and the like.

In another aspect of the invention, the cells are transformed with a vector that encodes for a fluorescent protein. Examples include, green fluorescent protein, enhanced green fluorescent protein, red fluorescent protein.

In another preferred embodiment, the invention provides a composition comprising: isolated cells, a vector encoding for a bispecific antibody and a fluorescent protein, wherein the isolated cells are transformed with the vector. Preferably the isolated cell is a stem cell and the vector is further comprised of oligonucleotides encoding complementary mRNA to specific target mRNA which codes for cell surface antigens. These cell surface antigens, are for example, MHC molecules, or any other molecule that has been identified as being involved in the disease process. The cell whereby the antigen is not expressed, is referred to as an “antigen depleted cell” or in the case of MHC molecules as an “MHC depleted cell”. Essentially, this method is used for depleting any antigen that is involved in a disease process.

In accordance with the invention, the bispecific antibody, targets the cell to a specific location in vivo. For example, the location can be to myocardial tissues, hepatocyte, kidneys and the like. The bispecific antibody determines the specific antigen to which the isolated cell is targeted. Preferably the cell is a stem cell so once the cell homes to the organ or tissue, the location of which is monitored using the methods discussed in detail in the examples which follow, the microenvironment of the target area induces the differentiation of the stem cell thereby the organ is repopulated with new cells. If the cell is antigen depleted and the antigen involved in the disease process is not present then the disease state is ameliorated. For example, MHC antigens.

Other aspects of the invention are described infra.

### BRIEF DESCRIPTION OF THE FIGURES

FIG. 1 is a graph from a flow cytometry assay showing the expression of VCAM-1 positivity by C166 cells by binding of anti-VCAM-1 to C166 cells. The line marked “isotype control” shows the isotype control antibody that was exposed to the C166 cells. The line marked “anti-VCAM-1” shows the positive expression of VCAM-1 on the C166 cells as detected by the anti-VCAM-1.

FIG. 2 is a photograph showing the non-aggregation of unarmed Lin-Sca+ cells in an aggregation assay.

FIG. 3 is a photograph showing the clear aggregation of Lin-Sca+ cells armed with anti-c-kit×anti-VCAM-1 at an arming dose of 500 ng/million in an aggregation assay.

FIG. 4 is a graph from flow cytometry data. The 6 panels show that as few as 12,500 armed ATC per ml mixed with 5.58 million normal white cells in the gate (total WBC 5.58×10³/mm³ from the CBC) can be detected by flow cytometry using this method. In Panel F, we show that as low as 0.4% ATC armed with OKT3×Herceptin/million could be detected in whole blood.

FIG. 5 shows phenotyping on the peripheral blood that was obtained Pre, 10 mins, 30 mins, 1 hr, 4 hrs, 10 hrs, and 24 hrs after an infusion of 2.0×10⁹ OKT3×9184 (anti-HER2/neu, a gift from Nexell) armed ATC. The kinetic study showed that armed ATC can be detected as ˜1% of the total WBC gate as soon as 30 mins after infusion, disappeared around 4 hrs, reappeared and persist in the circulation between 10 hrs and 24 hrs after the infusion.

### DETAILED DESCRIPTION OF THE INVENTION

The invention provides methods for the targeting and tracking of stem cells to specific locations within an animal's body. The methods used herein are useful in the therapeutic applications of repairing or colonizing specifically targeted areas within an animal, with stem cells, which then differentiate into mature cells of the specific cell type of the targeted area.

In a preferred embodiment, the method of the invention comprises arming stem cells with antibodies specific to antigens in a desired target area and arming the stem cells with a second antibody for the in vivo tracking of the stem cell from any area in the animal's body to the desired target area.

In accordance with the invention, stem cells from a patient are harvested, sorted, purified and identified. The stem cells are then armed with an antibody which will target the stem cell to the targeted location As an illustrative example, which is not meant to limit or construe the invention in any way, stem cells are targeted to myocardial tissue and by means of a second antibody are tracked to the heart tissue. These methods are fully described in the examples which follow. By way of illustration, to identify armed stem cells derived from any source and to identify their targeting to a desired location, stem cells were armed with anti-c-kit×anti-VCAM-1 to target injured myocardium. Preferably, Lin-Sca+ cells obtained an animal's bone marrow are purified by cell sorting after treatment with antibodies to on the MoFlo high-speed cell sorter. In brief, bone marrow cell suspensions are prepared by crushing the tibiae, femurs, and iliac crests of an animal, in Phosphate Buffered Saline (PBS) supplemented with 5% heat-inactivated Fetal Bovine Serum using a sterile mortar and pestle. Low density cells are separated and collected using density gradient centrifugation (1-step from Accurate Chemical). The mononucleated cells are collected and subjected to a cocktail of monoclonal antibodies. Examples include, but are not limited to including rat anti-Ter 119, B220, MAC-1, GR-1, Lyt-2, and L3T4 (PharMingen). These lineage positive cells are removed by magnetic bead depletion (Dynal). The lineage negative cells are suspended at a concentration of 3.0×10⁶ cells/ml and stained with goat anti-rat FITC at a final concentration of 0.5 μg/10⁶ cells. This allows sorting out of any Lin⁺ cells which were not depleted. Cells are incubated for 30 minutes on ice in the dark. Following a wash step in ice-cold buffer, the cells are then stained with anti-Scal PE at a final concentration of 1.0 μg/10⁶ cells. The cells are incubated in the dark on ice for 30 minutes, washed, cell strained and re-suspended at 20.0×10⁶ cells/ml. The cells are sorted by a high speed MoFlo based on their staining characteristics, PE⁺ positive (SCA⁺), FITC negative.

The unarmed Lin-Sca+ cells are treated with carboxyfluorescein diacetate N-succinimidyl ester (CFDA-SE) dye, washed and counted. CFDA-SE dye is used to evaluate the number of cell divisions that occur and be detected by gating for green fluorescence using flow cytometry. In order to obtain CFDA-SE marked armed Lin-Sca+ cells, cells are simultaneously labeled with CFDA-SE and armed with between about 50 ng to about 500 mg of bispecific antibody/million cells for 15 mins at 37° C. Preferably the cells are armed with about 50 ng of antibody/million cells, more preferably with about 100 ng antibody/million cells, most preferably with about 500 ng antibody/million cells. The arming process is complete after 15 mins at 37° C. About 150,000 cells can be armed with each arming dose of anti-c-kit×anti-VCAM-1 or anti-c-kit×isotope control. The cells are washed with about ten-fold dilution of volume of BSS containing 10% fetal calf serum and reconstituted in volumes adjusting the stem cells to 1×10⁶/ml. All arming doses are expressed as dose per million stem cells. Controls and experimental include but not limited to: 1) unarmed Lin-Sca+ cells, armed Lin-Sca+ (50 ng of anti-c-kit×anti-VCAM-1); 2) armed with Lin-Sca+ (100 ng of anti-c-kit×anti-VCAM-1); 3) armed Lin-Sca+ (500 ng of anti-c-kit×anti-VCAM-1); and 4) Lin-Sca+ armed with about 500 ng of irrelevant anti-c-kit×rat IgG2ₐ,6. FIG. 3 shows the clear aggregation of Lin-Sca+ cells armed with anti-c-kit×anti-VCAM-1 at an arming dose of 500 ng/million. There is similar binding at arming doses of 50 and 100 ng/million.

As an illustrative example, the following method is used to detect armed cells when administered to an animal. In preferred embodiments, the armed cells include but are not limited to lymphocytes, stem cells, NK cells or any other cell which can be used in the methods described herein. An example of a method for arming a cell with antibody is using flow cytometry. For example, about 10 μl per 0.5-2×10⁶ white blood cells (100 μl of whole peripheral blood) are used in the staining procedure. The staining procedure, includes for example, labeling of 12×75 mm tubes with the patients name and antibody to be added to the tube. Any number of controls can be sued. For example, controls for each specimen can be as follows:
a. “Control” (BD)=anti-mouse IgG1 FITC/IgG2 PE
b. “Leucogate” (BD)=CD45 FITC/CD14 PE.
c. Unarmed cells stained with anti-mouse IgG-2a.
   The samples are dispensed with syringe repeator pipette 100 μl of well mixed whole blood at the bottom of each tube, taking care to avoid contamination of the top of the tube with blood. The conjugated antibodies are added to the various tubes, near the bottom of the tube, using a micropipette. For example:
a. Add 20 μl of BD monoclonal antibodies to the “isotype” and “LeucoGate” tubes.
b. Add 10 μl of conjugated IgG-2a antibody to the designated test tubes.
   These are then mixed very gently in a vortex, followed by incubating the cell mixtures for 15 minutes, at room temperature, in the dark. Two ml of lysing solution is added (BD FACS Brand) to each tube and incubated in the dark for no more than 10 minutes. The tubes are then centrifuged at 1000 rpm, at room temperature, for 5 minutes. The supernatant is removed and cells are washed twice (2×) by adding 3 ml of PBS to each tube, mixed gently in a vortex and centrifuged at 1000 rpm for 5 minutes. The supernatants are again removed. The remaining drops of PBS wash buffer are removed by inverting and blotting the tubes onto absorbent paper. Into each tube 0.5 ml of 0.5% paraformaldehyde fixative solution is dispensed and the tubes are mixed gently by vortexing. They are then analyzed on a flow cytometer. For example, the percent of IgG-2a positive cells is based on the total CD45⁺ population. If analysis is not possible within 1 hour after fixation, cells are stored covered at 4° C. (refrigerator). Fixed cells are stable at this temperature for 4-5 days. Cells are acquired on a FACSCalibur (BD) flow cytometer using cellQUEST software (BD). Cells can be also analyzed using cellQUEST software: First cells are visualized by forward scatter (FSc) versus side scatter (SSc). The CD45 positive cells are then visualized and gated to eliminate debris. The IgG-2a positive cells are subsequently obtained as a percent of the CD45⁺ cells.

In accordance with the invention, this procedure can be used for different antigen specificities of armed cells such as T lymphocytes, e.g. Her2/neu, prostate tumor antigens, pancreatic tumor antigens, etc. Different isotypes of the arming antibody (e.g. IgG1, etc.) can be detected by utilizing secondary antibodies specific for the isotype. The secondary antibody can come from different sources, e.g. rat, sheep, goat etc; the important property being that it is targeted against the species of origin of the primary antibody. Also, secondary antibodies conjugated with different fluorochromes can be used, e.g. PE, FHTC, APC, etc.

In a preferred embodiment, the isolated and purified cells of the invention are used in functional assays to determine any properties of the cells. Depending on the isolated and purified cellular population, appropriate functional assays known in the art can be conducted. For example, if the population of cells are armed T cells specific for a desired antigen such as a tumor antigen, cytotoxic T cell assays, T cell proliferation assays, cytoline profiles, determination of surface antigens for T cell maturity or memory T cells, etc., can be carried out.

Isolation of cells useful in the present invention are well known in the art. For example, peripheral blood mononuclear cells (PBMCs) can be obtained from a subject and isolated by density gradient centrifugation, e.g., with Ficoll/Hypaque. Specific cell populations can be depleted or enriched using standard methods. For example, monocytes/macrophages can be isolated by adherence on plastic. T cells or B cells can be enriched or depleted, for example, by positive and/or negative selection using antibodies to T cell or B cell surface markers, for example by incubating cells with a specific primary monoclonal antibody (mAb), followed by isolation of cells that bind the mAb using magnetic beads coated with a secondary antibody that binds the primary mAb. Peripheral blood or bone marrow derived hematopoietic stem cells can be isolated by similar techniques using stem cell-specific mAbs (e.g., anti-CD34 mAbs). Specific cell populations can also be isolated by fluoresence activated cell sorting according to standard methods. Monoclonal antibodies to cell-specific surface markers known in the art and many are commercially available.

In another preferred embodiment, the methods of the invention are used for the repopulation of destroyed cells in an organ in need of repair. For example, kidneys, liver, heart, lungs, intestines and the like. The stem cells are armed with an antibody which is specific to the organ of interest. This is highly advantageous in patients suffering from organ damage due to trauma, car accidents, diseases, burn victims, patients with slow wound healing as in diabetes or hemophiliacs. In an illustrative example, stem cells are armed with antibodies that target, for example, regions of a damaged liver due to cirrhosis. The stem cells are also armed with a secondary antibody so that the trafficking of the cell is monitored. When the stem cells reach the liver or region of the liver in need of repair, the stem cells repopulate the region of the liver by developing into hepatic cells due to influences of the environment. More importantly, stem cells from closely match HLA donors can tolerize the immune system so the administered stem cells are not recognized as being “foreign.”

Any cell can be used in the methods of the invention, including but not limited to, stem cells, thymocytes, precursor cells and the like. A precursor cell population includes cells of a mesodermal derived cellular lineage, more particularly of hematopoietic lineage, endothelial lineage, muscle cell lineage, epithelial cell lineage and neural cell lineage.

A “precursor cell” can be any cell in a cell differentiation pathway that is capable of differentiating into a more mature cell. As such, the term “precursor cell population” refers to a group of cells capable of developing into a more mature cell. A precursor cell population can comprise cells that are totipotent, cells that are pluripotent and cells that are stem cell lineage restricted (i.e. cells capable of developing into less than all hematopoietic lineages, or into, for example, only cells of erythroid lineage). As used herein, the term “totipotent cell” refers to a cell capable of developing into all lineages of cells. Similarly, the term “totipotent population of cells” refers to a composition of cells capable of developing into all lineages of cells. Also as used herein, the term “pluripotent cell” refers to a cell capable of developing into a variety (albeit not all) lineages and are at least able to develop into all hematopoietic lineages (e.g., lymphoid, erythroid, and thrombocytic lineages). For example, a pluripotent cell can differ from a totipotent cell by having the ability to develop into all cell lineages except endothelial cells. A “pluripotent population of cells” refers to a composition of cells capable of developing into less than all lineages of cells but at least into all hematopoietic lineages. As such, a totipotent cell or composition of cells is less developed than a pluripotent cell or compositions of cells. As used herein, the terms “develop”, “differentiate” and “mature” all refer to the progression of a cell from the stage of having the potential to differentiate into at least two different cellular lineages to becoming a specialized cell. Such terms can be used interchangeably for the purposes of the present application.

As used herein, the term “population” refers to cells having the same or different identifying characteristics. The term “lineage” refers to all of the stages of the development of a cell type, from the earliest precursor cell to a completely mature cell (i.e. a specialized cell).

A stem cell population of the present invention is capable of developing into cells of mesodermal cell lineage, of ectodermal cell lineage or of endodermal cell lineage. As used herein, mesodermal cells include cells of connective tissue, bone, cartilage, muscle, blood and blood vessel, lymphatic and lymphoid organ, notochord, pleura, pericardium, peritoneum, kidney and gonad. Ectodermal cells include epidermal tissue cells, such as those of nail, hair, glands of the skin, the nervous system, the external sense organs (e.g, eyes and ears) and mucous membranes (such as those of the mouth and anus). Endodermal cells include cells of the epithelium such as those of the pharynx, respiratory tract (except the nose), digestive tract, bladder and urethra cells. Preferred cells within a stem cell population of the present invention include cells of at least one of the following cellular lineages: hematopoietic cell lineage, endothelial cell lineage, epithelial cell lineage, muscle cell lineage and neural cell lineage. Other preferred cells within a stem cell population of the present invention include cells of erythroid lineage, endothelial lineage, leukocyte lineage, thrombocyte lineage, erythroid lineage (including primitive and definitive erythroid lineages), macrophage lineage, neutrophil lineage, mast cell lineage, megakaryocyte lineage, natural killer cell lineage, eosinophil lineage, T cell lineage, endothelial cell lineage and B cell lineage.

Various techniques may be employed to separate the cells by initially removing cells of dedicated lineage. Monoclonal antibodies are particularly useful for identifying markers associated with particular cell lineages and/or stages of differentiation.

If desired, a large proportion of terminally differentiated cells may be removed by initially using a “relatively crude” separation. For example, magnetic bead separations may be used initially to remove large numbers of lineage committed cells. Desirably, at least about 80%, usually at least 70% of the total hematopoietic cells will be removed.

Procedures for separation may include but are not limited to, magnetic separation, using antibody-coated magnetic beads, affinity chromatography, cytotoxic agents joined to a monoclonal antibody or used in conjunction with a monoclonal antibody, including but not limited to, complement and cytotoxins, and “panning” with antibody attached to a solid matrix, e.g., plate, elutriation or any other convenient technique.

Techniques providing accurate separation include but are not limited to, flow cytometry, which can have varying degrees of sophistication, e.g., a plurality of color channels, low angle and obtuse light scattering detecting channels, impedance channels, etc.

In another preferred embodiment the stem cells may be transformed with DNA which codes for different growth factors and/or cytolines which will aid in the differentiation of the stem cells if the organ of interest is damaged to the extent that the microenvironment is not supportive of cell differentiation.

In another preferred embodiment, the stem cells are transformed with nucleic acids which are complementary to genes which code for antigens which are recognized by the immune system, such that these genes are rendered incapable of producing these antibodies. This is especially advantages in autoimmune diseases whereby the immune system recognizes self-antigens and mounts an immune reaction. Such autoimmune diseases include arthritis, myocarditis, myasthenia gravis and the like. These cells are then armed with antibodies which allows their targeting and detection at the site they home to. In accordance with the invention, the stem cells differentiate into mature cells representative of the target area but do not express the antigen which is inducing an autoimmune reaction thereby alleviating or down-regulating the autoimmune response. In other cases it is desirable to target the stem cells to areas whereby diseases such as cancer have destroyed certain target areas such as for example, colon cancer. Stem cells can be targeted to areas which have been removed by surgery or have been affected by chemotherapy and allowed to repopulate the area. In other cases, such as in hemophiliacs, it is desirable to target stem cells to the blood vessel lining thereby repairing the blood vessels and prevent further bleeding.

The methods of the invention have many advantages over gene therapy or organ transplantation, skin grafts and the like. The stem cells are immature and are able to repopulate without an immune response being mounted.

The terms, “patient”, “subject” or “animal” are used interchangeably and refer to a mammalian subject to be treated, with human patients being preferred In some cases, the methods of the invention find use in experimental animals, in veterinary application, and in the development of animal models for disease, including, but not limited to, rodents including mice, rats, and hamsters; and primates.

As used herein, the singular forms “a”, “an” and “the” include plural referents unless the context clearly dictates otherwise.

“Immune cells” as used herein, is meant to include any cells of the immune system that may be assayed, including, but not limited to, B lymphocytes, also called B cells, T lymphocytes, also called T cells, natural killer (NK) cells, lymphokine-activated killer (LAK) cells, monocytes, macrophages, neutrophils, granulocytes, mast cells, platelets, Langerhans cells, stem cells, dendritic cells, peripheral blood mononuclear cells, tumor-infiltrating (TIL) cells, gene modified immune cells including hybridomas, drug modified immune cells, and derivatives, precursors or progenitors of the above cell types.

“Activity”, “activation” or “augmentation” is the ability of immune cells to respond and exhibit, on a measurable level, an immune function. Measuring the degree of activation refers to a quantitative assessment of the capacity of immune cells to express enhanced activity when further stimulated as a result of prior activation. The enhanced capacity may result from biochemical changes occurring during the activation process that allow the immune cells to be stimulated to activity in response to low doses of stimulants.

Immune cell activity that may be measured include, but is not limited to, (1) cell proliferation by measuring the DNA replication; (2) enhanced cytoline production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

In accordance with the invention, T cells from patients are, preferably activated ex vivo either by soluble anti-CD3 antibody, or are co-activated by using anti-CD3 and anti-CD28 monoclonal antibodies, either by soluble or immobilized on a solid support A preferred solid support are plastics, or any surface upon which antibodies can be immobilized, or beads, such as, for example, Dynal beads. Once activated, T cells are armed with a bispecific antibody. The location and movements through the patient's body of these activated T cells can be monitored by using a labeled antibody that binds to a desired molecule on the surface of the activated T cell or directed to a portion of the bispecific antibody, such as for example, the F, region. Monitoring of the cells is achieved by using the flow cytometry methods of the invention. The T cells can also be labeled by agents which are detectable by any imaging techniques known in the art.

Bispecific antibodies are able to bind to the T cell receptor complex of the T cell with one binding arm and to tumor-associated antigens on the tumor cell with the second binding arm. Thereby, they activate T cells which kill tumor cells by releasing cytokines. Moreover, there is the possibility that T cells recognize tumor-specific antigens via their receptor during activation by bispecific antibodies and that, a long-lasting immunization is initiated. Of particular importance in this regard is the intact PC portion of the bispecific antibody which mediates the binding to accessory cells such as monocytes/macrophages/dendritic cells and causes these cells to become cytotoxic themselves and/or at the same time to transduce important co-stimulatory signals to the T cell.

In another preferred embodiment, it is desirable to track the T cells and verify that they have homed to desired regions. For example, an armed T cell which is targeted to tumor antigens such as, for example, Her2⁺ tumors, can be identified by labeling the T cell with a fluorescent marker, or secondary antibody that is detectable by flow cytometry or other methods well-known to one of ordinary skill in the art Examples of labels for detection of the armed T cells include but not limited to, green fluorescent proteins, avidins and the like. As an illustrative example, biopsies from a tumor to which tumor specific T cells armed with bispecific antibodies and labeled with a detectable marker, are used to confirm the presence of the armed T cells at the site of the tumor. The biopsied tissue is processed by methods well known in the art and prepared for use in cell detection assays. The preferred method is using the flow cytometry method as described in detail in the Examples which follow.

In another preferred embodiment, stem cells are harvested, transformed with complementary strand oligonucleotides which deletes certain gene segments (antisense therapy) good for diseases such as autoimmune diseases and the like. By decreasing the expression of the antigen that is recognized by cells involved in the autoimmune process.

Bispecific antibodies (BiAbs) have been used for targeting drugs, pro-drug activation, and immune recruitment strategies. They can directly mediate cytotoxicity to the tumor by specifically targeting the T cells to a tumor. BiAbs have been modified to bear enzymes for the conversions of circulating inactive pro-drug to active drug at the tumor site. Infused BiAbs have been used to recruit and redirect immune effector cells to target tumor cells in vivo, and/or arm effector T cells after ex vivo expansion for immunotherapy.

Armed T cells, bispecific antibodies and methods of activation have been described in U.S. Pat. No: 60/313,164, filed Aug. 17, 2001, which is incorporated herein in its entirety by reference. Briefly, T cells are activated and then armed with bispecific antibodies whose specificity is directed against a tumor antigen, like for example prostate tumors, prior to re-infusion into the patient Also, the cellular composition of the reinfusion is comprised of naive T and B cells and accessory cells such as dendritic cells which are powerful antigen presenting cells. Thus, these stimulate tumor specific T cell responses from the pool of naïve T cells.

Monoclonal antibodies directed to various markers on subpopulations of T lymphocytes have been used to activate immune effector cells. OKT3 antibody administered by injection, for example,.meets with CD3, and can cause a whole array of immune effects including release of IL-2, TNF-alpha, and/or IL-6, tissue damage, and either activation or suppression of T cell activity. It has also been shown by Weiner, Int. J. Cancer, Supplement 7, 63 (1992) that the action of the bispecific antibody is enhanced by co-administration of IL-2, so that combinational therapy resulted in management of a 100 to 1000 times greater tumor load tan with the anti-tumor monoclonal antibody alone. Alternatively, the co-stimulus observed in the use of the bispecific antibody may be provided through binding of the Fc domain of the antibody to the Fc monocyte receptor, which in turn provide the co-stimulus, possibly through binding of the B7 family of membrane proteins to CD28. Preactivation ex vivo of cytotoxic T cells with co-administration of bispecific F(ab′) has also been reported (Mezzanzanice, et al., Cancer Res., 51:5716 (1991)).

Monoclonal antibodies used in the production of, for example, targeting antibodies, bispecific antibodies, secondary antibodies used to monitor the location of any cell in the patient, are available from commercial sources, for example anti-CD3 monoclonal antibody (OKT3) is available from OrthoBiotech. Monoclonal antibodies specific for tumor antigens such as Her2⁺, (Herceptin®) can be purchased from Genentech, S.F., Calif.

Monoclonal antibodies may also be produced in the laboratory. Thus, in selecting an antibody specific for a common antigenic determinant displayed on the cell surface of cancers of a defined cell type, a mixture of cells is prepared, the mixture comprising cells from individual cell lines derived from a plurality of cancer cells of defined tissue type. This mixture of whole cells is then injected into a laboratory animal such as a mouse, according to a conventional immunization protocol to immunize the animal with the heterologous human tumor cells. Reactive B cells are then harvested from the animal, preferably the disrupted spleens, and fused with myeloma cells to form hybridomas. By maintaining the cell density below a critical level in which a statistical distribution function predicts one or two hybridomas per well, the likelihood of obtaining isolated single hybridomas is improved.

After cloning and outgrowth, supernatant medium containing the secreted monoclonal antibodies is removed. The screenings can then be carried out first by contacting the mixture of cancer cells of the defined tissue, and a mixture of cancer cells of a different tissue type, with the monoclonal antibody under conditions conducive to binding of the antibody to cells displaying the target antigenic determinant. A fluorescent dye that recognizes the antibody is then added and the cells are then evaluated in a flow cytometer to determine which cells have detectable dye and which do not. The cell types are distinguished by a log scale of emission light intensity. Thus, the cells are ranked into a first class having labeled antibody bound to the surfaces thereof and into a second class having no labeled antibody bound, thereby showing a bimodal distribution of cells in flow cytometry.

The second screen involves further screening tests on the cells showing a bimodal distribution in which individual cells of prostate cancer and other cells of cancer origin are labeled with the monoclonal antibody. Thus, each cancer cell type is individually tested with the labeled antibody to identify antibody with binding specificity for the cancer cells derived from the tissue of interest. Those antibodies which demonstrate unambiguous reactivity with, for example, prostate cancer-derived cells and no reactivity with nonprostate-derived cells are further tested. The cancer tissue types for which this method is intended in its therapeutic application include all those derived or arising from body organs unessential for viability such as ovary, breast, certain endocrine glands (thyroid), testicle, as well as prostate.

The third screening test is performed upon the monoclonal antibodies passing both the first and second screen, and involves determining the binding specificity of the labeled antibody for tissue sections derived from a plurality of cancers of defined cell type, together with controls of normal tissue sections from nonhomologous tissue.

The term “derived” as it applies herein means the cells were obtained by subculture of tumors isolated from patients. It also applies to cell lines established from non-solid tumors of the lymphatic system. The techniques for routine subculture of tumor cells are well known in the art, and include the use of growth factors, nutrients, support matrices; and hormones, as required for the particular tissue type. The techniques for immunization of experimental animals and subsequent cell fusion of splenic B cells to produce hybridomas, and their subsequent culture are conventional. The basic protocol utilized in the practice of the present invention is set forth in detail in Current Protocols in Immunology, vol. 1, J. E. Coligan, et al., eds., John Wiley & Sons: 1991, hereby incorporated by reference.

It is important in applying these protocols to the isolation of hybridomas according to the present invention, that a proper dilution of fused cells occurs, so that a substantial number of wells in the 96 well trays contain about 1-2 clones, and preferably, not more. At dilutions sufficiently great to attain this objective, some 6 to 12 percent of wells will contain 0 clones.

Screening by flow cytometry has several key advantages. First, it is important that a stable cell surface antigen be identified. By selecting only those antibodies that bind whole cells, the likelihood of choosing a stable surface component antigen is enhanced. The term “stable” means in the context of antibody/cellular interactions, that the target molecule is preferably a constitutive cell membrane glycoprotein integral to the structure and integrity of the membrane, and not a transient resident of the cell which is shed, displaced, or antigenically modified during the cell cycle.

A second advantage to screening by flow cytometry, is that the bimodal profile indicates that some cells bind the fluorophore labeled antibody and not others, which is a threshold indication of specificity. If only a single fluorescent peak is observed, this means that some antigen common to both the tumor cells and the non-prostate cells has been identified by the antibody. Two peaks mean that either one or more subsets of tumor cells have a unique antigen, one or more subsets of tumor cells but not all share an antigen with the non-tumor cells, or that the tumor cells have an antigen not shared by normal cells. Another advantage of this method of pre-selection is that the techniques of labeling cells and preparing them for flow cytometry are well known, and may be carried out routinely.

Bispecific antibodies have been utilized in a variety of therapeutic applications. U.S. Pat. No. 5,601,819 (Wong) discloses the use of a combinational CD3, and CD28 or interleukin 2 receptor bispecific antibody to selectively cause proliferation and destruction of specific T cell subsets. Belani, et al. showed that bispecific IgG functions in a B cell lymphoma model to retarget the specificity of T cells in low dose, and to cause nonspecific T cell activation with systemic cytoline production at higher doses. It was found that bsF(ab′)2 was also capable of retargeting T-cell mediated lysis by activated T cells. Thus, in many applications portions of antibodies, such as enzyme digested fragments, will mediate the effects otherwise observed for the intact antibody. These fragments necessarily contain the complementarity determining regions (CDRs) of the variable light and heavy chain antibody domains, and may be integrated with other protein fragments to form a bispecific antigen binding protein construct. This construct will minimally contain the CDRs including the interspersed constant framework beta sheet portions. These regions are easily identified following routine cloning and sequencing procedures, as disclosed in U.S. Pat. Nos. 5,530,101 and 5,585,089, hereby incorporated by reference. Cloning may be facilitated by PCR primers complementary to conserved sequences flanking the functional variable regions.

Useful bispecific antibodies combining a CDR specific for an effector cell and the CDR for a tissue specific antigen may also be humanized, either by replacing the light and heavy chain constant regions of the murine antibody with their human counterparts, or by grafting the CDRs onto a human antibody. Methods for carrying out these procedures are contained in U.S. Pat Nos. 5,530,101 and 5,585,089. The immune construct of the present invention may also be bispecific single chain antibodies, which are typically recombinant polypeptides consisting of a variable light chain portion covalently attached through a linker molecule to the corresponding variable heavy chain portion, as disclosed in U.S. Pat Nos. 5,455,030, 5,260,203, and 4,496,778, hereby incorporated by reference. A more complex construct for a single chain bispecific antibody also containing an Fc portion is provided in detail in U.S. Pat. No. 5,637,481. The principal advantage of constructs of this type is that only one species of antibody is produced, rather than three separate antibody types in the fused cell hybrid-hybridoma, which require further purification.

Other methods can be utilized in producing bispecific antibodies. A particular preferred method in the present invention is by chemical heteroconjugation of two monoclonal antibodies. Monoclonal antibodies, in addition to the described methods of production may be purchased from a commercial source. Chemical heteroconjugates can be created by the chemical linking of either intact antibodies or antibody fragments of different specificities. The preferred method for chemical heteroconjugation is described in the example section which follows.

In order that the invention may be more completely understood, several definitions are set forth. As used herein, the term “immunoglobulin” refers to a protein consisting of one or more polypeptides substantially encoded by immunoglobulin genes. The recognized immunoglobulin genes include the kappa, lambda, alpha, gamma (IgG₁, IgG₂, IgG₃, IgG₄), delta, epsilon and mu constant region genes, as well as the myriad immunoglobulin variable region genes. Full-length immunoglobulin “light chains” (about 25 KDa or 214 amino acids) are encoded by a variable region gene at the NH₂-terminus (about 110 amino acids) and a kappa or lambda constant region gene at the COOH-terminus. Full-length immunoglobulin “heavy chains” (about 50 KDa or 446 amino acids), are similarly encoded by a variable region gene (about 116 amino acids) and one of the other aforementioned constant region genes, e.g., gamma (encoding about 330 amino acids).

One form of immunoglobulin constitutes the basic structural unit of an antibody. This form is a tetramer and consists of two identical pairs of immunoglobulin chains, each pair having one light and one heavy chain. In each pair, the light and heavy chain variable regions are together responsible for binding to an antigen, and the constant regions are responsible for the antibody effector functions. In addition to antibodies, immunoglobulins may exist in a variety of other forms including, for example, Fv, Fab, and F(ab′)₂, as well as bifunctional hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423426 (1988), which are incorporated herein by reference). (See, generally, Hood et al., “Immunology”, Benjamin, N.Y., 2nd ed. (1984), and Hunkapiller and Hood, Nature, 323, 15-16 (1986), which are incorporated herein by reference).

An immunoglobulin light or heavy chain variable region consists of a “framework” region interrupted by three hypervariable regions, also called CDR's. The extent of the framework region and CDR's have been precisely defined (see, “Sequences of Proteins of Immunological Interest,” E. Kabat et al., U.S. Department of Health and Human Services, (1983); which is incorporated herein by reference). The sequences of the framework regions of different light or heavy chains are relatively conserved within a species. As used herein, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDR's. The CDR's are primarily responsible for binding to an epitope of an antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody may be joined to human constant segments, such as gamma 1 and gamma 3. A typical therapeutic chimeric antibody is thus a hybrid protein composed of the variable or antigen-binding domain from a mouse antibody and the constant or effector domain from a human antibody, although other mammalian species may be used.

As used herein, the term “humanized” immunoglobulin refers to an immoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor.” Constant regions need not be present, but if they are, they must be substantially identical to human immunoglobulin constant regions, i.e., at least about 85-90%, preferably about 95% or more identical. Hence, all parts of a humanized immunoglobulin, except possibly the CDR's, are substantially identical to corresponding parts of natural human immunoglobulin sequences. A “humanized antibody” is an antibody comprising a humanized light chain and a humanized heavy chain immunoglobulin. For example, a humanized antibody would not encompass a typical chimeric antibody as defined above, e.g., because the entire variable region of a chimeric antibody is non-human. One says that the donor antibody has been “humanized”, by the process of “humanization”, because the resultant humanized antibody is expected to bind to the same antigen as the donor antibody that provides the CDR's.

It is understood that the humanized antibodies may have additional conservative amino acid substitutions which have substantially no effect on antigen binding or other immunoglobulin functions. By conservative substitutions are intended combinations such as gly, ala; val, ile, leu; asp, glu; asn, gln; ser, thr; lys, arg; and phe, tyr.

Humanized immunoglobulins, including humanized antibodies, have been constructed by means of genetic engineering. Most humanized immunoglobulins that have been previously described have comprised a framework that is identical to the framework of a particular human immunoglobulin chain, the acceptor, and three CDR's from a non-human donor immunoglobulin chain.

A principle is that as acceptor, a framework is used from a particular human immunoglobulin that is unusually homologous to the donor immunoglobulin to be humanized, or use a consensus framework from many human antibodies. For example, comparison of the sequence of a mouse heavy (or light) chain variable region against human heavy (or light) variable regions in a data bank (for example, the National Biomedical Research Foundation Protein Identification Resource) shows that the extent of homology to different human regions varies greatly, typically from about 40% to about 60-70%. By choosing as the acceptor immunoglobulin one of the human heavy (respectively light) chain variable regions that is most homologous to the heavy“(respectively light) chain variable region of the donor immunoglobulin, fewer amino acids will be changed in going from the donor immunoglobulin to the humanized immunoglobulin. Hence, and again without intending to be bound by theory, it is believed that there is a smaller chance of changing an amino acid near the CDR's that distorts their conformation. Moreover, the precise overall shape of a humanized antibody comprising the humanized immunoglobulin chain may more closely resemble the shape of the donor antibody, also reducing the chance of distorting the CDR's.

Typically, one of the 3-5 most homologous heavy chain variable region sequences in a representative collection of at least about 10 to 20 distinct human heavy chains will be chosen as acceptor to provide the heavy chain framework, and similarly for the light chain. Preferably, one of the 1-3 most homologous variable regions will be used. The selected acceptor immunoglobulin chain will most preferably have at least about 65% homology in the framework region to the donor immunoglobulin.

In many cases, it may be considered preferable to use light and heavy chains from the same human antibody as acceptor sequences, to be sure the humanized light and heavy chains will make favorable contacts with each other. Regardless of how the acceptor immunoglobulin is chosen, higher affinity may be achieved by selecting a small number of amino acids in the framework of the humanized immunoglobulin chain to be the same as the amino acids at those positions in the donor rather than in the acceptor.

Humanized antibodies generally have advantages over mouse or in some cases chimeric antibodies for use in human therapy: because the effector portion is human, it may interact better with the other parts of the human immune system (e.g., destroy the target cells more efficiently by complement-dependent cytotoxicity (CDC) or antibody-dependent cellular cytotoxicity (ADCC)); the human immune system should not recognize the framework or constant region of the humanized antibody as foreign, and therefore the antibody response against such an antibody should be less than against a totally foreign mouse antibody or a partially foreign chimeric antibody.

The term “DNA construct” and “vector” are used herein to mean a purified or isolated polynucleotide that has been artificially designed and which comprises at least two nucleotide sequences that are not found as contiguous nucleotide sequences in their natural environment.

As used herein, the term “administering a molecule to a cell” (e.g, an expression vector, nucleic acid, a angiogenic factor, a delivery vehicle, agent, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

A cell has been “transformed”, “transduced”, or “transfected” by exogenous or heterologous nucleic acids when such nucleic acids have been introduced inside the cell. Transforming DNA may or may not be integrated (covalently linked) with chromosomal DNA making up the genome of the cell. In prokaryotes, yeast, and mammalian cells for example, the transforming DNA may be maintained on an episomal element, such as a plasmid. In a eukaryotic cell, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations (e.g., at least about 10).

Antibodies can also be genetically engineered. Particularly preferred are humanized immunoglobulins that are produced by expressing recombinant DNA segments encoding the heavy and light chain CDR's from a donor immunoglobulin capable of binding to a desired antigen, such as the human T cell CD3 complex, attached to DNA segments encoding acceptor human framework regions.

The DNA segments typically further include an expression control DNA sequence operably linked to the humanized immunoglobulin coding sequences, including naturally-associated or heterologous promoter regions. Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the humanized light chains, heavy chains, light/heavy chain dimers or intact antibodies, binding fragments or other immunoglobulin forms may follow (see, S. Beychok, Cells of Immunoglobulin Synthesis, Academic Press, New York, (1979), which is incorporated herein by reference).

Human constant region DNA sequences can be isolated in accordance with well known procedures from a variety of human cells, but preferably immortalized B-cells (see, Kabat op. cit. and WP87/02671). The CDR's for producing preferred immunoglobulins of the present invention will be similarly derived from monoclonal antibodies capable of binding to the predetermined antigen, such as the human T cell receptor CD3 complex, and produced by well known methods in any convenient mammalian source including, mice, rats, rabbits, or other vertebrates, capable of producing antibodies. Suitable source cells for the constant region and framework DNA sequences, and host cells for immunoglobulin expression and secretion, can be obtained from a number of sources, such as the American Type Culture Collection (“Catalogue of Cell Lines and Hybridomas,” sixth edition (1988) Rockville, Md., U.S.A., which is incorporated herein by reference).

Other “substantially homologous” modified immunoglobulins to the native sequences can be readily designed and manufactured utilizing various recombinant DNA techniques well known to those skilled in the art. For example, the framework regions can vary at the primary structure level by several amino acid substitutions, terminal and intermediate additions and deletions, and the like. Moreover, a variety of different human framework regions may be used singly or in combination as a basis for the humanized immunoglobulins of the present invention. In general, modifications of the genes may be readily accomplished by a variety of well-known techniques, such as site-directed mutagenesis (see, Gillman and Smith, Gene, 8, 81-97 (1979) and S. Roberts et al., Nature, 328,731-734 (1987), both of which are incorporated herein by reference).

Substantially homologous immunoglobulin sequences are those which exhibit at least about 85% homology, usually at least about 90%, and preferably at least about 95% homology with a reference immunoglobulin protein.

Alternatively, polypeptide fragments comprising only a portion of the primary antibody structure may be produced, which fragments possess one or more immunoglobulin activities (e.g., complement fixation activity). These polypeptide fragments may be produced by proteolytic cleavage of intact antibodies by methods well known in the art, or by inserting stop codons at the desired locations in vectors known to those skilled in the art, using site-directed mutagenesis.

As stated previously, the DNA sequences can be expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors contain selection markers, e.g., tetracycline or neomycin resistance, to permit detection of those cells transformed with the desired DNA sequences (see, e.g., U.S. Pat. No. 4,704,362, which is incorporated herein by reference).

E. coli is one prokaryotic host useful particularly for cloning the DNA sequences of the present invention. Other microbial hosts suitable for use include bacilli, such as Bacillus subtilus, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda The promoters will typically control expression, optionally with an operator sequence, and have ribosome binding site sequences and the like, for initiating and completing transcription and translation.

Other microbes, such as yeast, may also be used for expression. Saccharomyces is a preferred host, with suitable vectors having expression control sequences, such as promoters, including 3-phosphoglycerate kinase or other glycolytic enzymes, and an origin of replication, termination sequences and the like as desired.

In addition to microorganisms, mammalian tissue cell culture may also be used to express and produce the polypeptides of the present invention (see, Winnacker, “From Genes to Clones,” VCH Publishers, New York, N.Y. (1987), which is incorporated herein by reference). Eukaryotic cells are actually preferred, because a number of suitable host cell lines capable of secreting intact immunoglobulins have been developed in the art, and include the CHO cell lines, various COS cell lines, HeLa cells, preferably myeloma cell lines, etc, and transformed B-cells or hybridomas. Expression vectors for these cells can include expression control sequences, such as an origin of replication, a promoter, an enhancer (Queen et al., Immunol. Rev., 89, 49-68 (1986), which is incorporated herein by reference), and necessary processing information sites, such as ribosome binding sites, RNA splice sites, polyadenylation sites, and transcriptional terminator sequences. Preferred expression control sequences are promoters derived from immunoglobulin genes, SV40, Adenovirus, cytomegalovirus, Bovine Papilloma Virus, and the like.

The vectors containing the DNA segments of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts. (See, generally, Maniatis et al., Molecular Cloning: A Laboratory Manual, Cold Spring Harbor Press, (1982), which is incorporated herein by reference.)

Once expressed, the whole antibodies, their dimers, individual light and heavy chains, or other immunoglobulin forms of the present invention, can be purified according to standard procedures of the art, including ammonium sulfate precipitation, affinity columns, column chromatography, gel electrophoresis and the like (see, generally, R. Scopes, “Protein Purification”, Springer-Verlag, N.Y. (1982)). Substantially pure immunoglobulins of at least about 90 to 95% homogeneity are preferred, and 98 to 99% or more homogeneity most preferred, for pharmaceutical uses. Once purified, partially or to homogeneity as desired, the polypeptides may then be used therapeutically (including extracorporeally) or in developing and performing assay procedures, immunofluorescent staining, and the like. (See, generally, Immunological Methods, Vols. I and II, Lefkovits and Pernis, eds., Academic Press, New York, N.Y. (1979 and 1981)).

A preferred pharmaceutical composition of the present invention comprises the use of the monoclonal antibodies, which are either commercially available or produced by the methods described above. The monoclonal antibodies are preferably chemically heteroconjugated to produce the bispecific antibody of interest. These bispecific antibodies are then used to “arm” the activated T cells.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin Remington's Pharm. Sci., 15th Ed. (Mack Publ. Co., Easton (1975)).

As used herein, “arming” is the binding of the bispecific antibody portion specific for the T cell antigen of interest, that is the T cell receptor complex antigens such as CD3 and/or CD3 and CD28. The second portion of the bispecific antibody is the antibody which is specific for the tumor antigen of choice, thereby targeting the activated T cell to the specific tumor antigen.

The vectors may also comprise oligonucleotides which express mRNA which is complementary to mRNA coding for surface antigens which are involved, in for example, autoimmune diseases. Transforming cells, which are armed and labeled, with these vectors results in a decrease of such surface antigens, or “antigen depleted” or “MHC depleted” cells and these cells are targeted to specific tissues or organs wherein an autoimmune response is leading to the destruction of the cells comprising the tissue or organ. The antigen depleted cells, once they home to and differentiate into the mature cell type of that tissue or organ, will have decreased surface expressions of the antigens thereby decreasing the autoimmune response. This is advantageous over gene therapy methods, as the cells are autologous, the cells used can be stem cells which will differentiate into the mature cell of the targeted tissue type once they have trafficked or homed to the desired location and they can be tracked using the methods of the invention described infra.

Examples of antigens involved in autoimmunity are the MHC or HLA antigens. As used herein, the term “transplantation antigen” is used to refer to antigenic molecules that are expressed on the cell surface of transplanted cells, either at the time of transplantation, or at some point following transplantation. Generally these antigenic molecules are proteins and glycoproteins. The primary transplantation antigens are products of the major histocompatibility complex (MHC), located on chromosome 6 in humans. The human MHC complex is also called the human leukocyte antigen (HLA) complex. MHC antigens are divided into MHC class I antigens (in humans, this class includes HLA-A, -B, and -C antigens) and MHC class II antigens (in humans, this class includes HLA-DP, -DQ, and -DR antigens). Thus, the terms “MHC-II antigens”, “MHC class II antigens”, and “MHC class II transplantation antigens” are used interchangeably herein to refer to the class of proteins, which in humans, includes HLA-DP, -DQ and -DR antigens. While the terms “MHC class II genes” and “MHC-II genes” are used interchangeably herein to refer to the genes which encode the MIC class II transplantation antigens. The tern “MHC-II” is used herein to refer to the gene locus which encodes the MHC class II transplantation antigens, as well as the group of proteins encoded by that locus. Transplantation antigens also include cell surface molecules other than MHC class I and II antigens. These antigens include the following: (1) the ABO antigens involved in blood cell recognition; (2) cell adhesion molecules such as ICAM, which is involved in leukocyte cell-cell recognition; and (3) β2-microglobulin, a polypeptide associated with the 44 kd heavy chain polypeptide that comprises the HLA-I antigens but is not encoded by the MHC complex.

As used herein, the term “transplantation antigen nucleotide sequence” refers to nucleotide sequences associated with genes encoding transplantation antigens. Nucleotide sequences associated with genes include the region of the gene encoding the structural product, including intron and exon regions, and regions upstream of the structural gene associated with transcription, transcription initiation (including transcription factor binding sites), translation initiation, operator and promoter regions, ribosome binding regions, as well as regions downstream of the structural gene, including termination sites. Nucleotide sequences associated with genes also include sequences found on any form of messenger RNA (mRNA) derived from the gene, including the pre-mRNA, spliced mRNA, and polyadenylated mRNA.

As used herein, the term “MHC-II-depleted cell” refers to cells that are in some way depleted in the expression of at least one MHC class II transplantation antigen. This depletion may be manifested by a reduced amount of antigen present on the cell surface. Preferably, at least about 50%, more preferably about 80%, and even more preferably about 90% of the MHC-II antigen is eliminated at the cell surface. Most preferably, this depletion results in essentially total absence of the antigen at the cell surface. The amount of MEC-II antigens on the cell surface can be determined by a number of techniques known in the art. Usually such techniques make use of an antibody that specifically binds to the MHC-II antigen, wherein the antibody is linked to a compound that is easily quantified, such as a radionuclide, enzyme (e.g., horseradish peroxidase), or fluorescent dye. The techniques for measuring the amount of MHC-II antigens on the cell surface can be performed on living cells or on dead, fixed cell or tissue samples. Preferably the techniques are performed on living cells using a fluorescence-activated cell sorting analysis and fluorescently tagged antibodies that specifically bind to an MHC-II antigen.

As used interchangeably herein, the terms “oligo-nucleotides”, “polynucleotides”, and “nucleic acids” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, all as described herein.

The phrase “having a length of N bases” or “having a length of N nucleotides” is used herein to describe lengths along a single nucleotide strand, of a nucleic acid molecule, consisting of N individual nucleotides.

As used herein, the term “bind”, refers to an interaction between the bases of an oligonucleotide which is mediated through base-base hydrogen bonding. One type of binding is “Watson-Crick-type” binding interactions in which adenine-thymine (or adenine-uracil) and guanine-cytosine base-pairs are formed through hydrogen bonding between the bases. An example of this type of binding is the binding traditionally associated with the DNA double helix.

The term “complementary” and “complementary oligonucleotide” are used herein to refer to oligonucleotides or portions of polynucleotides which are capable of forming Watson-Crick-type binding interactions with another particular oligonucleotide or particular region of a polynucleotide. Generally, unless otherwise noted, the use of the term “complementary” means that all of the bases of the shorter of the two nucleotides, or portions of the nucleotides being discussed, are capable of Watson-Crick-type binding to a particular region of the other longer, or equal sized, oligonucleotide.

As used herein, “molecule” is used generically to encompass any vector, antibody, protein, drug and the like which are used in therapy and can be detected in a patient by the methods of the invention. For example, multiple different types of nucleic acid delivery vectors encoding different types of genes which may act together to promote a therapeutic effect, or to increase the efficacy or selectivity of gene transfer and/or gene expression in a cell. The nucleic acid delivery vector may be provided as naked nucleic acids or in a delivery vehicle associated with one or more molecules for facilitating entry of a nucleic acid into a cell. Suitable delivery vehicles include, but are not limited to: liposomal formulations, polypeptides; polysaccharides; lipopolysaccharides, viral formulations (e.g., including viruses, viral particles, artificial viral envelopes and the like), cell delivery vehicles, and the like.

As used herein, the term “oligonucleotide” refers to a polynucleotide formed from naturally occurring bases and pentofuranosyl groups joined by native phosphodiester bonds. This term effectively refers to naturally occurring species or synthetic species formed from naturally occurring subunits or their close homologs. The term “oligonucleotide” may also refer to moieties which function similarly to naturally occurring oligonucleotides but which have non-naturally occurring portions. Thus, oligonucleotides may have altered sugar moieties or intersugar linkages. Exemplary among these are the phosphorothioate and other sulfur-containing species which are known for use in the art. In accordance with some preferred embodiments, at least some of the phosphodiester bonds of the oligonucleotide have been substituted with a structure which functions to enhance the ability of the compositions to penetrate into the region of cells where the RNA or DNA whose activity to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with other structures which are, at once, substantially non-ionic and non-chiral, or with structures which are chiral and enantiomerically specific. Persons of ordinary skill in the art will be able to select other linkages for use in practice of the invention.

Oligonucleotides may also include species which include at least some modified base forms. Thus, purines and pyrimidines other than those normally found in nature may be so employed. Similarly, modifications on the pentofuranosyl portion of the nucleotide subunits may also be effected, as long as the essential tenets of this invention are adhered to. Examples of such modifications are 2′-O-alcyl- and 2′-halogen-substituted nucleotides. Some specific examples of modifications at the 2′ position of sugar moieties which are useful in the present invention are OH, SH, SCH₃, F, OCH₃, OCN, O(CH₂)ₙNH₂ or O(CH₂)ₙCH₃ where n is from 1 to about 10, and other substituents having similar properties.

As used herein, the terms “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasid cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

As used herein, “allogeneic” is used to refer to immune cells derived from non-self major histocompatibility complex donors. HLA haplotypes/allotypes vary from individual to individual and it is often helpful to determine the individual's HLA type. The HLA type may be determined via standard typing procedures and the peripheral blood lymphocytes (PBLs) purified by Ficoll gradients.

As will be recognized by those in the art, the term “host compatible” or “autologous” cells means cells that are of the same or similar haplotype as that of the subject or “host” to which the cells are administered.

As used herein, “partially-mismatched HLA”, refers to HLA types that are between about 20 to 90% compatible to the host's HLA type.

For many years, immunologists have hoped to raise specific cytotoxic cells targeting viruses, retroviruses and cancer cells. While targeting against viral diseases in general may be accomplished in vivo by vaccination with live or attenuated vaccines, no similar success has been achieved with retroviruses or with cancer cells. Moreover, the vaccine approach has not had the desired efficacy in immunosuppressed patients.

Chemokines and cytokines play a powerful role in the development of an immune response. The role of chemokines in leukocyte trafficking is reviewed by Baggiolini (1998) Nature 392:565-8, in which it is suggested that migration responses in the complicated trafficking of lymphocytes of different types and degrees of activation will be mediated by chemokines. The use of small molecules to block chemokines is reviewed by Baggiolini and Moser (1997) J. Exp. Med. 186:1189-1191.

The role of various specific chemokines in lymphocyte homing has been previously described. For example, Campbell et al. (1998) Science, showed that SDF-1 (also called PBSF), 6-C-line (also called Exodus-2), and MIP-3β (also called ELC or Exodus-3) induced adhesion of most circulating lymphocytes, including most CD4⁺ T cells; and MIP-3α (also called LARC or Exodus-1) triggered adhesion of memory, but not naive, CD4⁺ T cells. Tangemanm et al. (1998) J. Immunol. 161:6330-7 disclose the role of secondary lymphoid-tissue chemokine (SLC), a high endothelial venule (HEV)-associated chemokine, with the homing of lymphocytes to secondary lymphoid organs. Campbell et al. (1998) J. Cell Biol 141(4):1053-9 describe the receptor for SLC as CCR7, and that its ligand, SLC, can trigger rapid integrin-dependent arrest of lymphocytes rolling under physiological shear.

Several different ways, to assess maturity and cell differentiation, are available. For example, one such method is by measuring cell phenotypes. The phenotypes of immune cells and any phenotypic changes can be evaluated by flow cytometry after immunofluorescent staining using monoclonal antibodies that will bind membrane proteins characteristic of various immune cell types.

A second means of assessing cell differentiation is by measuring cell function. This may be done biochemically, by measuring the expression of enzymes, mRNA's, genes, proteins, or other metabolites within the cell, or secreted from the cell. Bioassays may also be used to measure functional cell differentiation or measure specific antibody production directed at a patient's tumor, tumor cell lines or cells from fresh tumors.

As used herein, “fresh tumors” refer to tumors removed from a host by surgical or other means.

In accordance with the invention, stem cells are detected using for example, bispecific antibodies comprised of anti-c-kit×anti-VCAM-1. The c-kit proto-oncogene encodes a transmembrane tyrosine kinase receptor for an unidentified ligand and is a member of the colony stimulating factor-1 (CSF-1)—platelet-derived growth factor (PDGF)—kit receptor subfamily. c-kit was shown to be allelic with the white-spotting (W) locus of the mouse. Mutations at the W locus affect proliferation and/or migration and differentiation of germ cells, pigment cells and distinct cell populations of the hematopoietic system during development and in adult life. The effects on hematopoiesis are on the erythroid and mast cell lineages as well as on stem cells, resulting in a macrocytic anemia which is lethal for homozygotes of the most severe W alleles, and a complete absence of connective tissue and mucosal mast cells. W mutations exert their effects in a cell autonomous manner, and in agreement with this property, c-kit RNA transcripts were shown to be expressed in targets of W mutations (Nocka, K., Majumder, S., Chabot, B., Ray, P., Cervone, M., Bernstein, A. and Besmer, P. (1989) Genes & Dev. 3, 816-826.). High levels of c-kit RNA transcripts were found in primary bone marrow derived mast cells and mast cell lines. Somewhat lower levels were found in melanocytes and eryrhroid cell lines.

The identification of the ligand for c-kit is of great significance and interest because of the pleiotropic effects it might have on the different cell types which express c-kit-and which are affected by W mutations in vivo. The demonstration of identity of c-kit with the W locus implies a function for the c-kit receptor system in various aspects of melanogenesis, gametogenesis and hematopoiesis during embryogenesis and in the adult animal.

The ligand of the c-kit receptor, KL, has been identified and characterized, based on the known function of c-kit/W in mast cells (Zsebo, K. M., et al., (1990a) Cell 63, 195-201; Zsebo, K. M., et al., (1990B) Cell 63, 213-214). The c-kit receptor in hematopoiesis KL stimulates the proliferation of bone marrow derived and connective tissue mast cells and in erythropoiesis, in combination with erythropoietin, KL promotes the formation of erythroid bursts (day 7-14 BFU-E). Furthermore, recent in vitro experiments with KL have demonstrated enhancement of the proliferation and differentiation of erythroid, myeloid and lymphoid progenitors when used in combination with erythropoietin, GM-CSF, G-CSF and EL-7 respectively suggesting that there is a role for the c-kit receptor system in progenitors of several hematopoietic cell lineages.

As used herein, c-kit ligand protein and polypeptide encompasses both naturally occurring and recombinant forms, i.e., non-naturally occurring forms of the protein and the potypeptide which are sufficiently identically to naturally occurring c-kit to allow possession of similar biological activity. Examples of such polypeptides includes the polypeptides designated KL-1.4 and S-KL, but are not limited to them. Such protein and polypeptides include derivatives and analogs. In one embodiment of this invention, the purified mammalian protein is a murine protein. In another embodiment of this invention, the purified mammalian protein is a human protein.

In another preferred embodiment of the invention, patient cells such as for example, stem cells, lymphocytes and the like can be modified to either express certain molecules or the suppression of expression of certain molecules, such as for example, antigens involved in autoimmune diseases. Stem cells may also be modified to express any receptor desired, such as for example, a modified T cell receptor, which would be advantageous in autoimmune diseases, or targeting of tumors, or antigens of infectious disease organisms such as viruses and the like. Hematopoietic stem cell specific regulatory elements are known in the art. Preferably regulatory elements derived from the CD34 gene are used (see e.g., Satterthwaite, A. B. et al. (1992) Genomics 12:788-794; Bur, T. C. et al. (1992) Blood 80:3051-3059).

Also described and provided herein are recombinant T cell receptor (TCR) constructs suitable for use in transducing the cells. Any cells may be used including stem cells, T cells, B cells and the like. Suitable constructs and uses thereof are described in International application no. US94/10033, which is hereby incorporated herein by reference. The recombinant TCR can be put under the control of a T cell specific promoter so that it is only expressed in T cells. For example, the promoter could be Granzyme A or Granzme B, which would cause the recombinant TCR to be expressed predominantly in NK cells and cytotoxic T lymphocytes (CTLs). Cytotoxic lymphocytes require Granzyme B for the rapid induction of DNA fragmentation and apoptosis in allogeneic target cells. Heusel et al. (1994) Cell 76:977-987. The T-cells derived from transduced cells-should home and circulate properly since they have matured in vivo and have not been directly manipulated subsequently ex vivo. They can then be expanded in number by administering cytokines in vivo. Since primarily antigen-activated cells proliferate in response to cytokines, modified T cells recognizing the target antigen should be relatively amplified. Also, it may be possible to get a stronger response from the T cells derived from the transduced cells. If more mature T cells are transduced with the recombinant TCR, they may have a dampened response if they are “memory” cells (i.e. previously exposed to antigens) and, therefore, “biased.”

Another advantage to genetically modified progenitor cells over mature T cells would be the ability to express the recombinant TCR in more than one hematopoietic lineage. For example, since macrophages are known to have the ability to engulf tumor cells, it may be useful to express-the recombinant TCR in macrophages.

The constructs can be prepared in a variety of conventional ways. Numerous vectors are now available which provide the desired features, such as long terminal repeats, marker genes, and restriction sites, which may be further modified by techniques known in the art. The constructs can encode a signal peptide sequence in addition to the antigenic specificity region and cytoplasmic signalling sequence, to ensure that the recombinant TCR is properly processed post-translationally and expressed on the cell surface. Preferably, the construct is under the control of a T cell specific promoter. Suitable T cell specific promoters include, but are not limited to, Granzyme A, Granzyme B and CD8.

In one embodiment, the signal transducing region and antigenic specificity region are both obtained from TCRs (“classic TCR”). In another embodiment the constructs encode chimeric polypeptides comprising the signal transducing region obtained from a T cell specific receptor or the Fcγ receptor and an antigen binding portion of an immunoglobulin or of a NK receptor (“chimeric TCR”).

The recombinant classic TCRs are functional, preferably full length, TCRα and γ, or γ and δ, polypeptides which have been derived from a T cell with known antigenic specificity. Suitable sources of antigen-specific receptors include, but are not limited to, cytotoxic T lymphocytes, T helper cells and NK cells. In another embodiment, the polypeptides may be recombined so as to form a single functional polypeptide with the Vα and Vβ regions forming the antigen binding site. In another embodiment, the Vα and Vβ regions from different TCRs may be recombined to endow the TCR with a different specificity.

The T cell progeny of the cells containing the recombinant classic TCR polypeptides are “MHC restricted”, that is, they will only recognize antigen in the presence of MHC. Thus, when using these cells to treat a patient, the TCRs must be able to recognize the same haplotype as that of the host. It is well within the skill of one in the art to determine if the haplotype of the host will be compatible with a particular TCR. The classic TCR approach is advantageous where the antigen is expressed as a short peptide on the cell surface by processing internally and presented in the groove of an MHC molecule.

In the case of the chimeric TCR, the chimeric molecule contains an antigen binding sequence from an antibody or another receptor, a transmembrane sequence and a sequence that can transduce a signal and elicit a function. A variety of these and related molecules have been cloned and expressed in various T cell lines. Kuwana et al. (1987) Biochem. Biophys. Res. Comm. 149:960-968; Gross et al. (1989) Trans. Proc. 21:127-130; Becker et al. (1989) Cell 58:911-921; Gross et al. (1989) Proc. Natl. Acad. Sci. USA 86:10024-10028; and Goverman et al. (1990) Cell 60:929-939. Several chimeric TCRs have been created and found to be active in targeting T cells to the antigen recognized by the antibody binding site. Eshhar (1993) Proc. Natl. Acad. Sci. USA 90:720-724; and Hwu et al. (1993) J. Exp. Med. 178:361-366.

Suitable signal transducing regions can be obtained from receptors that have activation capacity through a specific chain including, but not limited to, the γ chain of the F_{C} receptor, the CD3 ζ chain, IL-2 receptor γ chain, CD8 or CD28. Alternatively, the antigen binding domain may be associated with TCR α or β chain constant regions, which will transduce signal via association with endogenous CD3 ζ chain. Preferably, the functional portion of the chimeric molecule is the signaling region of a F_{c}γ or ζ polypeptide and the antigen binding domain is a variable region of an antibody. The variable region may be either the V_{H} or V_{L} regions or, preferably, a single chain recombinant thereof. Methods for recombination in mammalian cells may be found in Molecular Cloning, A Laboratory Manual (1989) Sambrook, Fritsch and Maniatis, Cold Spring Harbor, N.Y.

The T cell progeny of the cells containing the chimeric TCR molecules, recognize antigen in the absence of MHC when the antigen binding site is derived from an antibody and thus may not be MHC restricted. These molecules are suitable for use in all hosts regardless of haplotype.

Upon reintroduction of the genetically modified cells into the host and subsequent differentiation, T cells are produced that are specifically directed against the specific antigen. Generally, suitable antigens include those found on specific cancer cells. More specifically, suitable antigens include, but are not limited to, viral coat proteins and specific surface proteins of cancer cells.

In accordance with the invention, the stem cells are removed, armed with the desired bispecific antibodies for targeting and tracking of the stem cells to the desired location. A second antibody or label may used for monitoring the location of the stem cells. In many situations, cell immunotherapy involves removal of bone marrow or other source of hematopoietic cells from a human host, isolating the progenitor cells from the source and optionally expanding the isolated cells. Meanwhile, the host can be treated to partially, substantially or completely ablate native hematopoietic capability. The isolated cells can be modified during this period of time, so as to provide for cells having the desired genetic modification. In the case of complete hematopoietic ablation, stem cell augmentation is also be required. After completion of the treatment of the host, the modified cells-may then be restored to the host to provide for the new capability. The methods of hematopoietic cell removal, host ablation and stem/progenitor cell repopulation are known in the art. If necessary, the process may be repeated to ensure the substantial repopulation of the modified cells. The present invention is advantageous in that it provides for the specific targeting and monitoring of cells in vivo.

The modified cells may be administered in any physiologically acceptable vehicle, normally intravascularly, although they may also be introduced into bone or other convenient site where the cells may find an appropriate site for regeneration and differentiation (e.g., thymus). Usually, at least about 1×10⁵ cells are administered, preferably 1×10⁶ or more. The cells may be introduced by injection, catheter, or the like. If desired, although not required, factors may also be included, including, but not limited to, interleukins, e.g. IL-2, IL-3, IL-6, and IL-11, as well as the other interleukins, the colony stimulating factors, such as GM-CSF, interferons, e.g. γ-interferon, erythropoietin.

Immune cells express a variety of cell surface molecules which can be detected with either monoclonal antibodies or polyclonal antisera. Immune cells that have undergone differentiation or activation can also be enumerated by staining for the presence of characteristic cell surface proteins by direct immunofluorescence in fixed smears of cultured cells.

The following definitions are used throughout the application:

The term “fluorescent component” or “fluorescent label” or “labeled” refers to a component capable of absorbing light and then re-emitting at least some fraction of that energy as light over time. The term includes discrete compounds, molecules, naturally fluorescent proteins and macro-molecular complexes or mixtures of fluorescent and non-fluorescent compounds or molecules. The term “fluorescent component” or “fluorescent label” also includes components that exhibit long lived fluorescence decay such as lanthanide ions and lanthanide complexes with organic ligand sensitizes, that absorb light and then re-emit the energy over milliseconds. Other labels include different fluorochromes and fluorescent proteins such as green fluorescent protein. Fluorochromes which may find use in a multicolor analysis include phycobiliproteins, e.g., phycoerythrin and allophycocyanins; fluorescein and Texas red.

Activated T Cells (ATC) are a heterogeneous population of human lymphocytes predominantly T lymphocytes of CD8 phenotype that have been triggered to proliferate after stimulation with OKT3 and grown in low doses of IL-2. ATC has been safe given in combination with subcutaneous or continuous infusion of IL-2 at low doses of 300,000 IU/m²/day and GM-CSF at doses as high as 250 μg/m²/day or as low as 125 μg/m²/twice weekly. ATC may also be administered with other immune augmenting cytokines or chemokines, such as for example, IL-12.

Murmonab OKT3: OKT3 (OrthoClone OKT3) has been extensively characterized in both preclinical and clinical testing. OKT3 is a murine IgG2a MAb directed at human CD3 and is commercially available from OrthoBiotech, Raritan, N.J. It is purchased in 5 mg vials containing 5 ml of bacteriostatic water. It is used to produce ATC, coat Dynal beads for the production of COACTS, and produce chemical heteroconjugate with 9184 or Herceptin®.

IL-2 Proleukin: Proleukin (recombinant IL-2) is purchased from Chiron, Emeryville, Calif. It is approved for the treatment of renal cell carcinoma. The clinical grade drug is used to expand T cells in culture. It is currently being used in this laboratory to expand ATC.

Murm 9.3 is a mouse MAb of the IgG2a isotype directed at the CD28 receptor on human T cells. The 9.3 MAb was produced under clinical grade condition by Abbott-Biotech. The antibody is coimmobilized with OKT3 on Dynal beads to activate T cells during culture. The antibody is not infused into patients. The monoclonal antibody is removed when the beads are removed.

The BiAb: anti-c-kit×anti-VCAM-1 or any combinations thereof, to target and monitor cell locations within a patients body. In the case of armed T cells, BiAbs also include, but not limited to OKT3×9184 or OKT3×Herc [either designated as Her2Bi] are used to arm ATC so that the non-MHC restricted cytotoxicity exhibited by ATC can be redirected by the bispecific antibody to lyse targets expressing a specific target antigen. ATC armed with Her2bi are designated ATC armed with Her2Bi. All antibodies, used herein are of clinical grade. For example, clinical grade 9184 MAb: Clinical material, GOMP 9184 (anti-Her-2/neu), is supplied in vials containing 1 mg by Nexell Corporation, Irving, Calif. 9184 has been extensively characterized by Nexell and has been used for purging of stem cell products in the European market.

Anti-Her2/neu monoclonal antibody (9184, Nexell Corporation). Anti-Her2 (9184 monoclonal provided as a study drug by Nexell Corporation is a murine IgG1 monoclonal antibody directed at Her2/neu. The binding characteristics and its ability to mediate redirected cellular cytotoxicity towards MCF-7, SK-BR-3 and PC-3 have been well documented in our preclinical studies.

Her2/neu is a tumor associated antigen (TAA) on prostate cancers. Her2/neu (Her2) belongs to the epidermal growth factor receptor family of tyrosine kinases. The Her2/neu oncogene encodes an 185 kDa transmembrane receptor with significant sequence homology to class I receptor tyrosine kinase family. Her2 is over-expressed in breast, ovarian, lung, gastric, oral, and prostate cancers. The over-expression of Her2 makes it an ideal target. The expression of Her2/neu on prostate cancer, however, is controversial. There are reports that suggest that expression of Her2/neu on prostate cancers is low or absent, or high.

Herceptin®: Herceptin® (Genentech, SF, Calif.) has been extensively characterized in preclinical and clinical trials. The MAb is commercially available for in vivo use for the treatment of stage IV breast cancer in combination with Taxol.

OKT3: Clinical grade OKT3 obtained commercially will be heteroconjugated to 9184 or Herceptin® to produce OKT3×9184 or OKT3×Herc, respectively.

Armed T Cells (The “Drug”):

Armed ATC are ATC grown for 6-14 days and armed with Her2Bi (OKT3×9184 or OKT3×Herc).

For certain therapeutic applications, a DNA expression vector encoding a desired cytokine, such as, for example, IL-12, chemokine, or any other immune-augmenting molecule of the invention can be introduced into immune cells of the present invention such as, for example, T cells.

Armed stem cells of the invention include antibodies that target the stem cells to a desired location. However, in certain therapeutic applications, an expression vector encoding a desired tissue specific molecule, such as VCAM-1, can be used to transform a stem cell.

The term “vector” as used herein (including “expression vector”) means any nucleic acid sequence of interest capable of being incorporated into a host cell resulting in the expression of a nucleic acid of interest Vectors can include, e.g., linear nucleic acid sequences, plasmids, cosmids, phagemids, and extrachromosomal DNA. Specifically, the vector can be a recombinant DNA. Also used herein, the term “expression”, or “gene expression”, is meant to refer to the production of the protein product of the nucleic acid sequence of interest, including transcription of the DNA and translation of the RNA transcript.

Vectors can be constructed which also comprise a detectable/selectable marker gene. In preferred embodiments these marker genes are fluorescent proteins such as green fluorescent protein (GFP), cyan-(CFP), yellow-(YFG), blue-(BFP), red-(RFP) fluorescent proteins; enhanced green fluorescent protein (EGFP), EYFP, EBFP, Nile Red, dsRed, mutated, modified, or enhanced forms thereof, and the like.

As used herein, the “green-fluorescence protein” is a gene construct which in transfected or infected cells, respectively, shines green under ultraviolet light and thus enables the detection of a cell transfected or infected, respectively, with GFP in a simple manner.

These can include vectors, liposomes, naked DNA, adjuvant-assisted DNA, gene gun, catheters, etc. Vectors include chemical conjugates such as described in WO 93/04701, which has a targeting moiety (e.g. a ligand to a cellular surface receptor), and a nucleic acid binding moiety (e.g. polylysine), viral vector (e.g. a DNA or RNA viral vector), fusion proteins such as described in PCT/US95/02140 (WO 95/22618) which is a fusion protein containing a target moiety (e.g. an antibody specific for a target cell) and a nucleic acid binding moiety (e.g. a protamine), plasmids, phage etc. The vectors can be chromosomal, non-chromosomal or synthetic.

Preferred vectors include viral vectors, fusion proteins and chemical conjugates. Retroviral vectors include Moloney murine leukemia viruses. DNA viral vectors are preferred. Viral vectors can be chosen to introduce the cytokine or chemokine to cells of choice. Such vectors include pox vectors such as orthopox or avipox vectors, herpesvirus vectors such as herpes simplex I virus (HSV) vector (Geller, A. I. et al, J. Neurochem., 64:487(1995); Lim, F., et al., in DNA Cloning: Mammalian Systems, D. Glover, Ed. (Oxford Univ. Press, Oxford, England) (1995); Geller, A. I. et al., Proc. Natl. Acad. Sci. USA 87:1149 (1990)) Adenovirus vectors (LeGal LaSalle et al., Science, 259:988 (1993); Davidson, et al., Nat. Genet. 3:219 (1993); Yang et al., J. Virol. 69:2004 (1995)) and Adeno-associated virus vectors (Kaplitt, M. G. et al., Nat. Genet. 8:148 (1994)).

Pox viral vectors introduce the gene into the cells cytoplasm. Avipox virus vectors result in only short term expression of the nucleic acid. Adenovirus vectors, adeno-associated virus vectors and herpes simplex virus vectors are preferred for introducing the nucleic acid into neural cells. The adenoviras vector results in a shorter term expression (about 2 months) than adeno-associated virus (about 4 months), which in turn is shorter than HSV vectors. The vectors can be introduced by standard techniques, e.g. infection, transfection, transduction or transformation. Examples of modes of gene transfer include for example, naked DNA calcium phosphate precipitation, DEAE dextran, electroporation, protoplast fusion, lipofection, cell microinjection and viral vectors.

Uses of green fluorescent protein for the study of gene expression and protein localization are well known. The compact structure makes GFP very stable under diverse and/or harsh conditions such as protease treatment, making GFP an extremely useful reporter in general.

New versions of green fluorescent protein have been developed, such as a “humanized” GFP DNA, the protein product of which has increased synthesis in mammalian cells. One such humanized protein is “enhanced green fluorescent protein” (EGFP). Other mutations to green fluorescent protein have resulted in blue-, cyan- and yellow-green light emitting versions.

Endogenously fluorescent proteins have been isolated and cloned from a number of marine species including the sea pansies Renilla reniformris, R. kollikeri and R. mullerei and from the sea pens Ptilosarcus, Stylatula and Acanthoptilum, as well as from the Pacific Northwest jellyfish, Aequorea Victoria; Szent-Gyorgyi et al. (SPIE conference 1999), D. C. Prasher et al., Gene, 111:229-233 (1992) and several species of coral (Matz et al. Nature Biotechnology, 17 969-973 (1999). These proteins are capable of forming a highly fluorescent, intrinsic chromopbore through the cyclization and oxidation of internal amino acids within the protein that can be spectrally resolved from weakly fluorescent amino acids such as tryptophan and tyrosine.

As used herein, the term “antibody” refers to a polypeptide or group of polypeptides which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as wells as fragments, including Fab, Fab′, F(ab)₂, and F(ab′)₂ fragments.

As used herein, an “antigenic determinant” is the portion of an antigen molecule that determines the specificity of the antigen-antibody reaction. This term will be used interchangeably with the term “target antigen”. An “epitope” refers to an antigenic determinant of a polypeptide. An epitope can comprise as few as 3 amino acids in a spatial conformation which is unique to the epitope. Generally an epitope consists of at least 6 such amino acids, and more usually at least 8-10 such amino acids. Methods for determining the amino acids which make up an epitope include x-ray crystallography, 2-dimensional nuclear magnetic resonance, and epitope mapping e.g. the Pepscan method described by H. Mario Geysen et al. 1984. Proc. Natl. Acad. Sci. U.S.A. 81:3998-4002; PCT Publication No. WO 84/03564; and PCT Publication No. WO 84/03506.

Antibodies directed against surface antigens for detection of tumors and the like membrane proteins can also be fluorescently labeled. Specificity for particular cell types is likely to be easier to achieve with antibodies than with other molecules because antibodies can be raised against nearly any surface marker. Also, microinjected antibodies could label sites on the cytoplasmic face of the plasma membrane, blood vessels and the like.

In a preferred embodiment, armed and labeled ATC are used in the tracking and treatment of hormone refractory prostate cancer (HRPC) and other patients with Her2⁺ tumors. However, the present invention allows for the tracking and treatment of all types of tumors as a bispecific antibody can be generated with specificity for a tumor and/or tissues, organs and the like, present in an individual patient. The present invention is advantageous in that it allows for use of autologous T cells and monoclonal antibody generation for a specific antigen present in an individual patient, thus allowing the re-direction of a cell to a desired target location. Thus, treatment can be tailored for each individual patient and allows for changes in treatment if the tumor antigen changes, by generation of new monoclonal antibodies against the new tumor antigen. The new tumor specific antibody can then be heteroconjugated to the T cell activating antibody to form a new bispecific antibody, specific for the new tumor antigen and then used to arm the autologous ATC and reinfused into the patient.

The above procedure is an example of how the treatment protocol is used for patients with hormone refractory prostate cancer, but can be modified according to the type of tumor to be treated. Tho amount of armed ATC administered to the patient will also vary depending on the condition of the patient and should be determined via consideration of all appropriate factors by the practitioner. Preferably, however, about 1×10⁶ to about 1×10¹², more preferably about 1×10⁸ to about 1×10 ¹¹, and even more preferably, about 1×10⁹ to about 1×10¹⁰ armed ATC cells are utilized for adult humans. These amounts win vary depending on the age, weight, size, condition, sex of the patient, the type of tumor to be treated, the route of administration, whether the treatment is regional or systemic, and other factors. Those skilled in the art should be readily able to derive appropriate dosages and schedules of administration to suit the specific circumstance and needs of the patient.

Methods of Reintroducing cellular components are known in the art and include procedures such as those exemplified in U.S. Pat. No. 4,844,893 to Honsik, et al. and U.S. Pat. No. 4,690,915 to Rosenberg. For example, administration of activated CD8⁺ cells via intravenous infusion is appropriate.

A “vector” is a composition which can transduce, transfect, transform or infect a cell, thereby causing the cell to express nucleic acids and/or proteins other than those native to the cell, or in a manner not native to the cell. A cell is “transduced” by a nucleic acid when the nucleic acid is translocated into the cell from the extracellular environment Any method of transferring a nucleic acid into the cell may be used; the term, unless otherwise indicated, does not imply any particular method of delivering a nucleic acid into a cell. A cell is “transformed” by a nucleic acid when the nucleic acid is transduced into the cell and stably replicated. A vector includes a nucleic acid (ordinarily RNA or DNA) to be expressed by the cell. A vector optionally includes materials to aid in achieving entry of the nucleic acid into the cell, such as a viral particle, liposome, protein coating or the like. A “cell transduction vector” is a vector which encodes a nucleic acid capable of stable replication and expression in a cell once the nucleic acid is transduced into the cell.

The invention has been described in detail with reference to preferred embodiments thereof. However, it will be appreciated that those skilled in the art, upon consideration of this disclosure, may make modifications and improvements within the spirit and scope of the invention. The following non-limiting examples are illustrative of the invention.

### EXAMPLES

Materials and Methods

Components used for ATC.

Orthoclone OKT3 (muromonab-CD3). OKT3 is purchased from Ortho-Biotech (Raritan, N.J.). OKT3 is supplied as a sterile solution in packages of 5 ampoules (NDC 59676-101-01) containing 5 mg of muromonab-CD3. Proleukin® (Aldesleukin, IL-2). Proleukin® (recombinant IL-2) is purchased from Chiron (Emeryville, Calif.). ATC are expanded in the presence of low dose IL-2 (100 IU/ml) in RPMI 1640 (Bio Whittaker) supplemented with 2-5% human serum (Bio Whittaker). RPMI 1640 (Bio Whittaker) is supplemented with 2 mM L-glutamine (Bio Whittaker), and 50 μg/ml gentimicin (Bio Whittaker).

“Armed ATC” are the best descriptive names for the armed and activated T cells. The chemically heteroconjugated anti-CD3×anti-Her2 (either OKT3×9184 or OKT3×Herceptin®) is referred to as Her2bi. Therefore, ATC armed with anti-CD3×anti-Her2/neu BiAb are designated Her2bi armed ATC. OKT3×Herceptin® has been abbreviated to OKT3×Herc.

Preparation of anti-CD3×anti-Her2 Bispecific Antibody.

Equimolar concentrations of OKT3 and anti-Her2 (9184 or Herceptin) are conjugated. OKT3 is reacted with Traut's reagent at room temperature (RI) for 1 hr and 9184, Herceptin, or control irrelevant antibodies are reacted with sulphosuccinimidyl 4-N-maleimidomethyl)cyclohexane-1 -carboxylate at RT. Both Mabs are purified on 10 DG columns (Biorad) in PBS to remove unbound cross linker. The cross-linked Mabs are mixed at equimolar ratios and cojugated at 4° C. overnight. At these concentrations, dimer formation is optimal and multimer formation is minimized. The reactants, products, and purified fractions of the heteroconjugation reaction are visualized by SDS non-reducing PAGE and Coomassie blue staining. The final products OKT3×9184 or OKT3×Herc are cleared for final use only after 7 days of bacteria and fungal cultures, PCR for mycoplasma (ATCC, Catalog #90-1001K), and assay for endotoxin (Bio Whittaker, Catalog #50-6470) are all negative.

Each bispecific antibody heteroconjugate lot (previous testing negative for bacteria, fungi, Mycoplasma, and endotoxin) is tested by a dose-titration arming of normal cryopreserved ATC or COACTS against PC-3, MCF-7, and/or SK-BR-3 target cells prior to release. Multiple vials of pretested normal donors have been expanded for 14 days, cryopreserved in aliquots for lot testing as well as providing normal controls in the evaluation of the armed patient ATC. Each lot must exhibit a dose-titration effect with increased specific cytotoxicity at the same E/T ratio when as the arming dose of the Her2Bi is increased. The arming dose range of each lot is determined by such a titration. The lot is rejected if a % specific cytotoxicity of at least about 50% of PC-3, MCF-7 or SK-BR-3 at an E/T of 25:1 can not be attained after a 20 hr cytotoxicity assay with an arming dose of 50 ng/million ATC.

Flow Cytometry for Detection of Cells.

Antibody Utilized to Detect Armed T Cells:

Source: Caltag laboratories, Burlingame, Calif.
Catalog No. M32204

Antibody: Affinity-isolated goat anti-mouse IgG-2a in phosphate buffered
saline (PBS).
Conjugated with phycoerythrin (R-PE)
Contains 0.1% azide as preservative.
Bovine serum albumin has been added as a stabilizing agent
Final protein concentration is 4-5 mg/ml.
Quantity utilized is 10 μl per 0.5-2×10⁶ white blood cells
(WBC; usually 100 μl of whole peripheral blood).
   Staining Procedure:

1. Label 12×75 mm tubes with the patients name and antibody to be added to the tube.
Include tubes for controls for each specimen as follows:
   a. “Control” (BD)=anti-mouse IgG1 FITC/IgG2 PE
   b. “Leucogate” (BD)=CD45 FITC/CD14 PE.
   c. Unarmed cells stained with anti-mouse IgG-2a.

2. Dispense with syringe repeator pipette 100 μl of well mixed whole blood at the bottom of each tube. Avoid contamination of the top of the tube with blood.

3. Add conjugated antibodies to the various tubes, near the bottom of the tube, using a micropipette.
a. Add 20 μl of BD monoclonal antibodies to the “isotype” and “LeucoGate” tubes.
b. Add 10 μl of conjugated IgG-2a antibody to the designated test tubes.

4. Mix very gently in a vortex.

5. Incubate the cell mixtures for 15 minutes, at room temperature, in the dark.

6. Add 2 ml of lysing solution (BD FACS Brand) to each tube and incubate in the dark for no more than 10 minutes.

7. Centrifuge tubes at 1000 rpm, at room temperature, for 5 minutes.

8. Remove supernatantly decanting into a beaker containing 10% bleach.

9. Wash cells 2× by adding 3 ml of PBS to each tube, mix gently in vortex and centrifuge at 1000 rpm for 5 minutes. Supernatants are decanted in a beaker containing 10% bleach

10. Remove the remaining drops of PBS wash buffer by inverting and blotting the tubes onto absorbent paper.

11. Dispense into each tube 0.5 ml of 0.5% paraformaldehyde fixative solution.

12. Mix gently by vortexing.

13. analyze on a flow cytometer.
The percent of IgG-2a positive cells is based on the total CD45+ population.
If analysis is not possible within 1 hour after fixation, store cells covered at 4 C (refrigerator). Fixed cells are stable at this temperature for 4-5 days.

14. Cells are aquired on a FACSCalibur (BD) flow cytometer using cellQUEST software (BD).

15. Cells are also anlayzed using cellQUEST software:
First cells are visualized by forward scatter (FSc) versus side scatter (SSc).
The CD45 positive cells are then visualized and gated to eliminate debris.
The IgG-2a positive cells are subsequently obtained as a percent of the CD45+ cells.
   General Comments:

This is procedure is tailored to different antigen specificities of armed T lymphocytes, e.g. Her2 Neu, prostate tumor antigens, pancreatic tumor antigens, etc.

Different isotypes of the arming antibody (e.g. IgG₁, etc.) can be detected by utilizing secondary antibodies specific for the isotype.

The secondary antibody can come from different sources, e.g. rat, sheep, goat etc; the important property being that it is targeted against the species of origin of the primary antibody.

Also, secondary antibodies conjugated with different fluorochromes can be used, e.g. PE, FITC, APC, etc.

Preparation, Culture, Arming and Cryopreservation of ATC or COACTS.

Peripheral blood mononuclear cells (PBMC) from normal subjects and cancer patients are isolated by Ficoll-Hypaque (Lymphoprep from Nycomed Pharma, Oslo, Norway). PBMC are activated on plates coated with 2 μg/ml immobilized OKT3 or 20 ng/ml of soluble OKT3 (Ortho Biotech, Inc., Raritan, N.J.). Unless otherwise indicated, ATC are grown for 14 days in RPMI 1640 medium (Bio Whittaker, Walkersville, Md.) supplemented with antibiotics, L-glutamine, 100 IU/ml of IL-2 (Chiron Corp., Emeryville, Calif.), and 10% fetal calf serum FCS (Hyclone, Logan, Utah) or human serum (Bio Whittaker) as indicated. Cells are counted and maintained at 10⁶/ml. Viability is determined by trypan blue exclusion. T cells are grown from normal subjects or cancer patients. The T cells are washed, counted, and incubated (“armed”) with BiAb at the indicated doses (10⁶ T cells for 1 hr at 4° C. in 125 μl of media). The T cells are washed twice in complete medium prior to testing.

Activation, Culture, Cryopreservation, Thawing, and Washing of ATC.

In brief, lymphocytes are obtained by leukopheresis, cultured at a density of 1-3×10⁶ cells/ml in RPMI 1640 medium supplemented with 2 mM of L-glutamine, 100 IU/ml of IL-2, 10-20 ng/ml of OKT3-, and 2% pooled human serum. Cells are cultured for a maximum of 14 days. The ATC are counted, split and/or fed every 34 days with complete medium based on cell concentration N6 additional OKT3 is added. After culture, ATC are harvested and washed using the Fenwal Cell harvester, and cryopreserved in 10% DMSO and 20% protein (albumin or autologous plasma) using controlled rate freezing and storage in liquid nitrogen. No exogenous IL-2, OKT3, or other culture reagents (e.g. medium components) are present in the final cryopreserved product

Initiation, Splitting, and Harvest of ATC Cultures.

PBMC at a concentration of 1×10⁶ mononuclear cells/ml are activated with 20 ng/ml of soluble OKT3 in RPMI 1640 supplemented with L-glutamine, gentimicin, 100 IU/ml of IL-2, and 2% human serur in Stericel Multiple Container Sets. The activated T cells are counted split, and fed based on their expansion rate. After 6-14 days of culture, the ATC are harvested. If the amount of ATC to be harvested is 1 to 10 liters, procedures well-known in the art will be used for small volume T cell harvest & Cryopreservation. For volumes exceeding 10 liters, the ATC are harvested using a Fenwal Cell Harvesting System. Cryopreservation of unarmed or armed ATC is conducted using commercial human serum.

Anti-CD3 (OKT3) and anti-CD28 (9.3) monoclonal antibodies immobilized on beads are used to cross link cellular receptors. OKT3 is purchased from Ortho-Biotech (Raritan, N.J.). 9.3 antibody, lot #3-309-900411 was produced for Dr. Carl June by Abbott Biotech. OKT3 and 9.3 are linked to paramagnetic, polystyrene Dynabeads via tosyl chemistry in a 1:1 stoichiometry as per the manufacturer's protocol. The magnetic beads and the monoclonal antibodies linked to the beads are all produced under GMP conditions for clinical use.

Infusion Product Free of Carryover Cytokines.

The infused cell product does not contain exogenous cytokines. Data show that the IL-2 used in culture is undetectable by ELISA after 1 wash of ATC in 50 ml tube. Before washing of the cultured ATC, 3.5 JU/ml of IL-2 was detected. In a second experiment, duplicate 50 ml tubes spiked with 1200, 600, and 300 IU of IL-2/ml had no detectable IL-2 after 1 wash. The ELISA is sensitive to 50 pg/ml (<1 IU/ml).

Components to Produce OKT3×9184 or OKT3×Herceptin®.

This application uses two BiAbs. Both BiAbs target Her2/neu use the chemical heteroconjugation process to produce the combination of anti-CD3×anti-Her2/neu.

OKT3×9184 consists of clinical grade Orthoclone OKT3 (muromonab, IgG2a murine Mab directed at CD3 is purchased from Ortho-Biotech, Raritan, N.J.) chemically heteroconjugated to clinical grade 9184 (IgG1 murine Mab directed at Her2/neu is a gift from Nexell, Corporation, Irving, Calif.).

OKT3×Herceptin® (OKT3×Herc) consists of clinical grade OKT3 (IgG2a murine monoclonal antibody directed at CD3) chemically heteroconjugated to clinical grade Herceptin® (trastzumab, a humanized IgG1 Mab directed at Her2/neu is purchased from Genentech, San Francisco, Calif.).

Traut's Buffer: Traut's Buffer (pH 8) consists of Triethanolamine (Sigma Ultra #T9534), 1.5 M NaCl (Sigma), 1 mM EDTA (Disodium Dihydrate) (Sigma)

Sulphosuccinimidyl 4-(N-maleimidomethyl) cyclohexane-1-carboxylate: Sulpho-SMCC (SMCC buffer, Pierce #22322) is purchased from Pierce, Rockford, Ill.

Heteroconjugation Products: Equimolar quantities of OKT3 crosslinked with Traut's buffer and Sulpho-SMCC crosslinked 9184, Herceptin, or Rituxan are incubated overnight at 4° C. A non-reducing SDS gel is performed to identify the presence of monomer, diner, or multimer. The material is sterile filtered and quality control by sterility, endotoxin, and Mycoplasma testing using standard procedures and release criteria (Limulus Amebocyte Lysate, Bio Whittaker Catalog #504470; Mycoplasma detection kit, ATCC, Catalog #90-1001K).

ATC Armed with OKT3×9184 or OKT3×Herc.

ATC are thawed after cryopreservation, armed with a pretitrated dose ranging from 5-100 ng of unpurified chemically heteroconjugated Her2Bi per million ATC, washed free of non-binding antibodies, and infused into the patients.

Arming of the Clinical Product.

From 3 to 10 billion (20% more than the targeted amount is processed to account for washing and processing losses) cryopreserved ATC or COACTS are thawed, washed, resuspended in plasmanate containing 2% albumin, and armed in centrifuge tubes by incubating for 1 hr at 4° C. The Her2Bi is washed twice in plasmanate containing 2% albumin, and transferred into a transfer bag for infusion into the patient. An aliquot is removed for viability, sterility and cytotoxicity testing.

Tumor Cell Lines and Monoclonal Antibodies.

The breast cancer lines MCF-7 cells and SK-BR-3 are purchased from ATCC Rockville, Md. OKT3 is purchased from OrthoBiotech. The 9184 is an anti-Her2/neu, IgG1 provided by Nexell. Herceptin® is purchased from Genentech, SF, Calif.), Rituxan (anti-CD20) is purchased from Genentech. T84.55 hybridoma (anti-carcinoembryonic antigen) is purchased from ATCC, Rockville, Md. IG3 (anti-prostate specific membrane antigen) is a gift from Alton Boynton of Northwest Biotherapeutics, Seattle, Wash.

### Example 1

### Trafficking of ATC in vivo and Accumulation at Tumor Sites

Blood phenotyping and biopsies of accessible metastatic lesions are obtained The biopsied tissue is used in indirect immunofluorescence assays using anti-IgG₂ₐ antibodies to detect the OKT3 component of OKT3×Herceptin®. The clinical protocol will incorporate biopsies when they are obtained.

### Example 2

### Confirmation of the Expression of VCAM-1 on C166 Cells

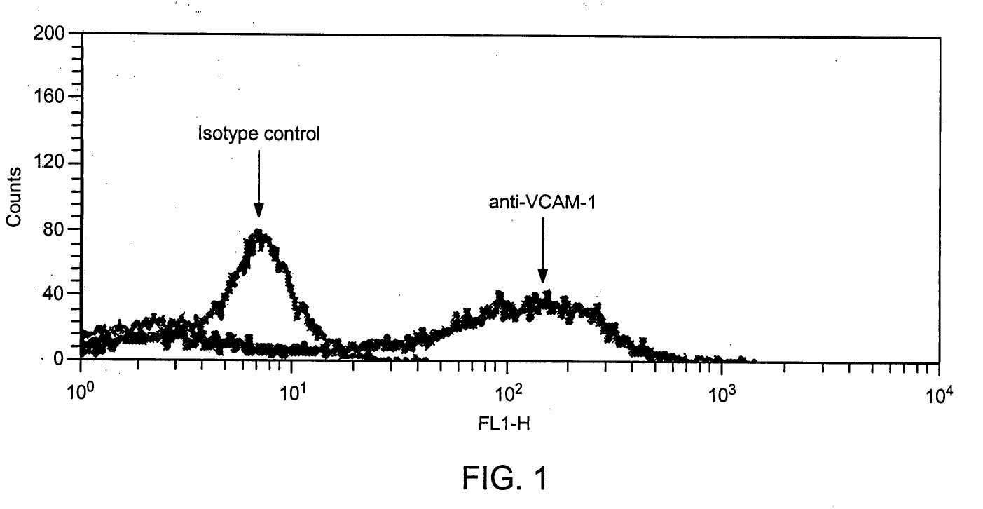

VCAM-1 is constitutively expressed on C166 cells (endothelial cell line purchased from ATCC). We confirmed the presence of VCAM-1 by performing binding of 1: g of anti-VCAM-1 (anti-CD106, 429MVCAM.A,rat IgG2ₐ6), (PharMingen) per million C166 cells detected using secondary goat anti-rat FITC conjugated antibody. The cells were incubated with antibody for 30 mins at 37° C., washed three times in DMEM containing 10% fetal calf serum. Flow cytometry was performed to determine the number and intensity of positive cells. FIG. 1 shows the expression of VCAM-1 positivity by C166 cells by binding of anti-VCAM-1 to C166 cells. The green line shows the isotype control antibody that was exposed to the C166 cells. The red line shows the positive expression of VCAM-1 on the C166 cells as detected by the anti-VCAM-1.

### Example 3

### Arming and Targeting of Stem Cells to Specific Locations in vivo

To identify armed stem cells derived from any source and to identify their targeting to a desired location, murine stem cells were armed with anti-c-kit×anti-VCAM-1 to target injured myocardium.

Heteroconjugation of Anti-c-kit×Anti-VCAM-1. Based on the expression of VCAM-1 on injured myocardium and other injured tissues, anti-c-kit×anti-VCAM-1 was produced. FIG. 1 shows the gel products resulting from the heteroconjugation reaction. Anti-c-kit (2 mg) in 50 mM NaCl, 1 mM EDTA, pH 8.0 was reacted with a 10 fold M excess of Traut's reagent (2-iminothiolane HCl, Pierce) and anti-VCAM-1 (2 mg) in 0.1 M sodium phosphate, 0.15 M NaCl at pH 7.2 was reacted with a 4 fold molar excess of sulphosuccinimidyl 4-(N-maleimidomethyl) cyclohexane-1-carboxylate (Sulpho-SMCC) at room temperature for 1 hr. Both BiAbs were purified on PD-10 columns (Pharmacia, Uppsala, Sweden) in Phosphate buffered saline (PBS) to remove unbound cross-linker. The cross-linked mAbs were mixed immediately at equimolar ratios and conjugated at 4° C. overnight. The gel in FIG. 1 shows the marker (lane 1), the parental anti-c-kit (lane 2), the parental anti-VCAM-1 (lane 3), and the heteroconjugated BiAb (lane 4) each loaded with 8 μg of protein. Densitometry of lane 4 containing antis-c-kit×anti-VCAM-1 showed 64% monomers, 17% dimers, and 19% multimers.

### Example 4

### Aggregation of Lin-Sca+ c-kit+ Cells with C166 Cells

Lin-Sca+ cells obtained C57BL/6 mice bone marrow (section 5.1.1) were purified by cell sorting after treatment with antibodies to on the MoFlo high-speed cell sorter (as described in section 5.1.6.) In brief, bone marrow cell suspensions are prepared by crushing the tibiae, femurs, and iliac crests of mice in Phosphate Buffered Saline (PBS) supplemented with 5% heat-inactivated Fetal Bovine Serum using a sterile mortar and pestle. Low density cells are separated and collected using density gradient centrifugation (1-step from Accurate Chemical). The mononucleated cells are collected and subjected to a cocktail of monoclonal antibodies, including rat anti-Ter 119, B220, MAC-1, GR-1, Lyt-2, and L3T4 (PharMingen). These lineage positive cells are removed by magnetic bead depletion (Dynal). The lineage negative cells are suspended at a concentration of 3.0×10⁶ cells/ml and stained with goat anti-rat FITC at a final concentration of 0.5 μg/10⁶ cells (this allows the sorting out any Lin⁺ cells which were not depleted). Cells are incubated for 30 minutes on ice in the dark. Following a wash step in ice-cold buffer, the cells are then stained with anti-Scal PE at a final concentration of 1.0 μg/10⁶ cells. The cells are incubated in the dark on ice for 30 minutes, washed, cell strained and re-suspended at 20.0×10⁶ cells/ml. The cells are sorted by a high speed MoFlo based on their staining characteristics, PE⁺ positive (SCA⁺), FITC negative.

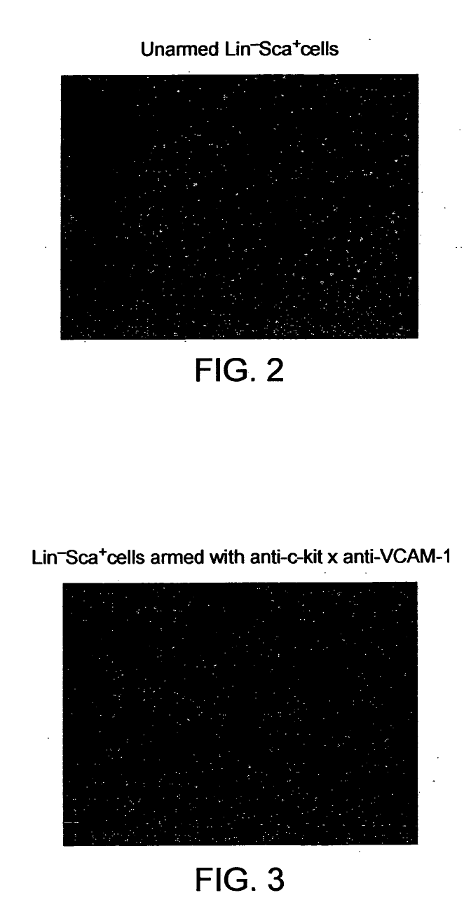

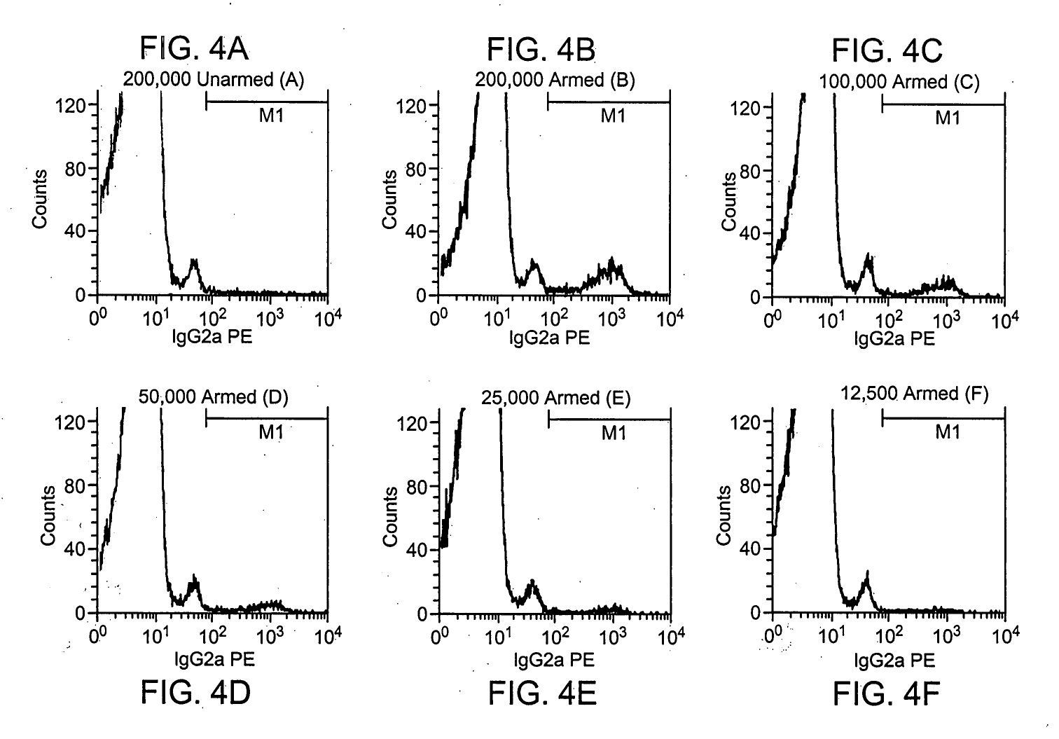

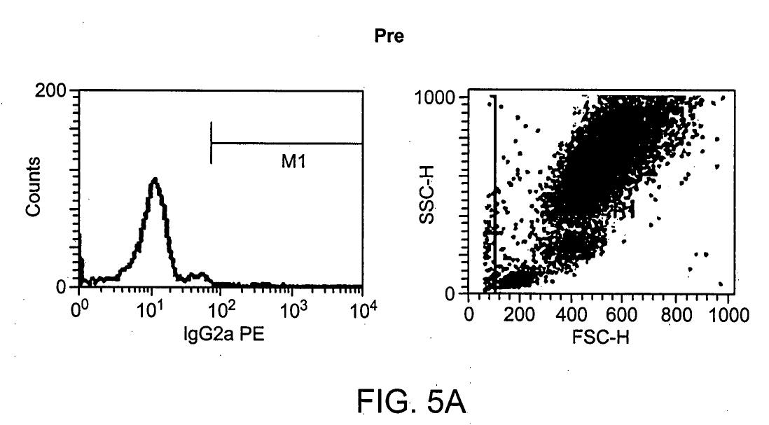

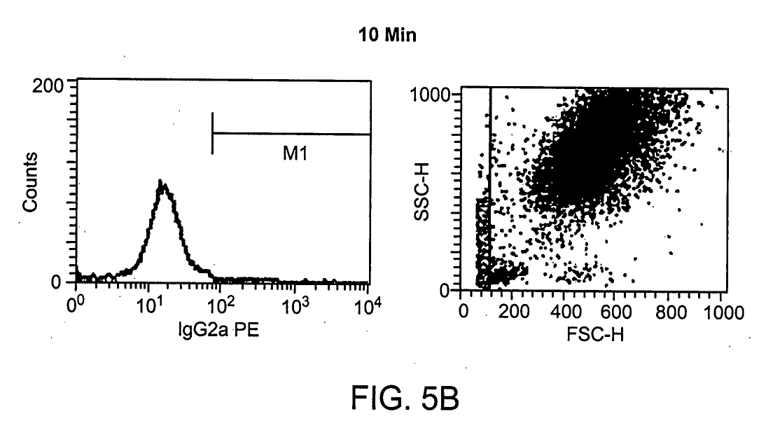

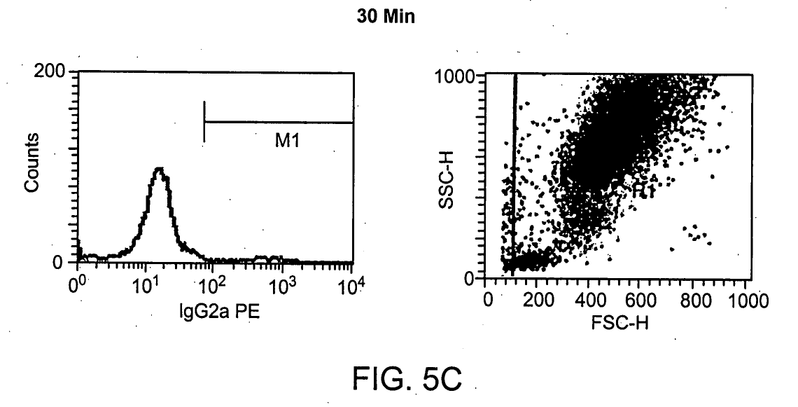

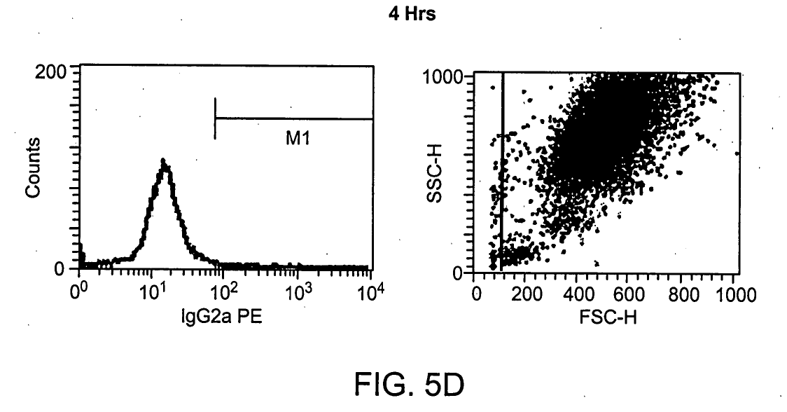

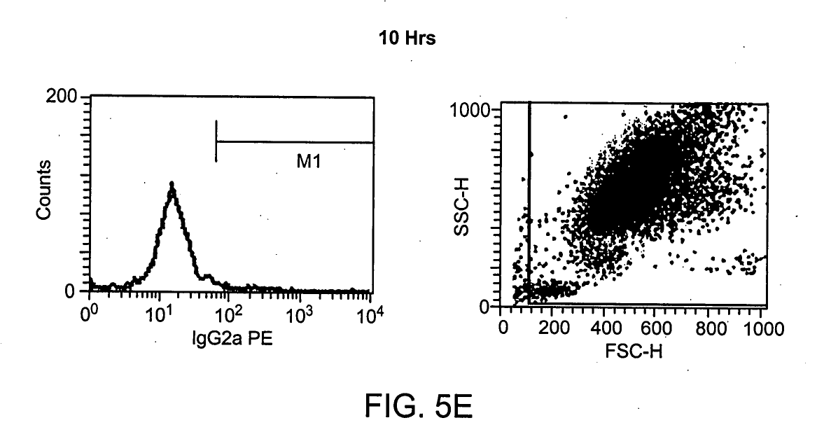

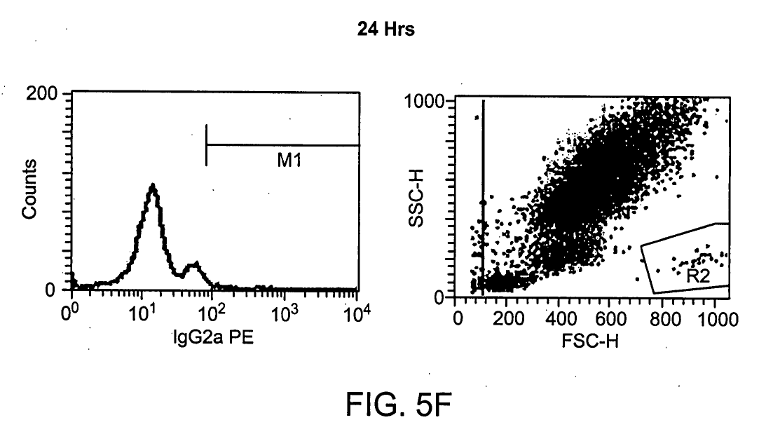

In order to perform the aggregation assay, the sorted Lin-Sca+ cells were aliquoted into unarmed and c-kit×anti-VCAM-1 armed cells. The unarmed Lin-Sca+ cells were treated with CFDA-SE, washed and counted for the aggregation assay. In order to obtain CFDA-SE marked armed Lin-Sca+ cells, which were simultaneously labeled with CFDA-SE and armed with 50 ng, 100 ng, and 500 ng of bispecific antibody/million cells for 15 mins at 37° C. Simultaneous CFDA-SE marking and bispecific antibody marking was done to conserve on the number of cells that are available after sorting. Pretitrated with the time and temperature for arming activated T cells showed that the arming process was complete after 15 mins at 37° C. Approximately 150,000 cells were armed with each arming dose of anti-c-kit×anti-VCAM-1 or anti-c-kit×isotope control. The cells were washed with a ten-fold dilution of volume of BSS containing 10% fetal calf serum and reconstituted in volumes adjusting the stem cells to 1×10⁶/ml. All arming doses are expressed as dose per million stem cells. The stem cells plated onto C166 cells were as follows: 1) unarmed Lin-Sca+ cells, armed Lin-Sca+ (50 ng of anti-kit×anti-VCAM-1); 2) armed with Lin-Sca+ (100 ng of anti-c-kit×anti-VCAM-1); 3) armed Lin-Sca+ (500 ng of anti-c-kit×anti-VCAM-1); and 4) Lin-Sca+ armed with 500 ng of irrelevant anti-c-kit×rat IgG2ₐ,6. The C166 cells were plated overnight so they would adhere to flat-bottomed microtiter wells in DMBM. The armed or unarmed stem cells were plated at a stem cell:C166 cell ratio of in the range of 5 to 10:1 and incubated overnight at 37° C. FIG. 2 shows the non-aggregation of unarmed Lin-Sca+ cells and FIG. 3 shows the clear aggregation of Lin-Sca+ cells armed with anti-c-kit×anti-VCAM-1 at an arming dose of 500 ng/million. There was similar binding at arming doses of 50 and 100 ng/million. Lin-Sca+ cells armed with irrelevant anti-c-kit×rat IgG^{2a},6 looked similar to FIG. 2.

### Example 5

### Aggregation of Lin-Sca⁺ Sorted Murine Stem Cells Coated with 500 ng/million of Chemically Heteroconjugated Anti-c-kit (rat IgGₖ)×anti-VCAM-1 (Rat IgGₖ)

FIG. 3 shows lin-Sca⁺ purified murine stem cells incubated overnight at 37° C. with a VCAM-1⁺ cell line, C166 (a murine endothelial cell lines that constitutively expresses murine VCAM-1). FIG. 2 shows the unarmed (uncoated) Lin-Sca⁺ cells do not aggregate with the C166 cell line.

What is claimed is:

1. A method for tracking cells in vivo, the method comprising the steps of:
(a) isolating and purifying stem cells from a subject;
(b) providing a chemically heteroconjugated bispecific antibody with a binding site specific for a stem cell antigen and a binding site specific for a target antigen in a patient; and,
(c) arming the stem cells with the bispecific antibody under conditions wherein;
(i) the bispecific antibody binds to the stem cells via the c-kit ligand; and,
(ii) the second antigenic binding site of the bispecific antibody is free to bind to the target antigen; and,
(iii) binding of a labeled antibody to the Fc region of the bispecific antibody; or,
(iv) fluorescently labeling the bispecific antibody thereby a secondary labeled antibody is not required; and,
(d) reinfusing the aimed and labeled stem cells into a patient; and,
(e) tracking the armed and labeled stem cells by extracting blood and/or tissue samples from the patient at different time intervals; and,
(f) identifying the armed and labeled cells by phenotyping the cells using flow cytometry cell sorting; and,
(g) identifying the armed and labeled cells by immunohistochemical staining or other methods to detect the primary antibody on the cells in various target tissues such as bone marrow, spleen, liver, pancreas, lungs, neural tissue, gastrointestinal track, heart, vascular endothelium.

2. The method of claim 1, wherein the bispecific antibody is specific for c-kit ligand of stem cells and myocardial antigens.

3. The method of claim 2, wherein the bispecific antibody is specific for myocardial VCAM-1, NCAM-1, PECAM.

4. The method of claim 1, wherein secondary antibody, binding to the Fc region of the bispecific antibody, is fluorescently labeled

5. The method of claim 1, wherein the bispecific antibody is directly fluorescently labeled.

6. The method of claim 1, wherein the armed and labeled stem cells home to, and bind to the target tissue antigens.

7. The method of claim 6, wherein the stem cells accumulate at the target antigen site.

8. The method of claim 7, wherein the stem cells differentiate into cells typical of the targeted tissue or organ.

9. The method of claim 1, wherein the patient sample is a blood sample.

10. The method of claim 1, wherein the patient sample is a biopsy of the targeted tissue or organ.

11. The method of claims 9 or 10, wherein the samples are subjected to flow cytometry cell sorting to identify the armed and labeled cells by phenotyping.

12. The method of claim 11, wherein the samples are taken at different time intervals after reinfusion of the stem cells to track the location of the armed and labeled cells.

13. The method of claim 12, wherein the numbers of armed and labeled cells at a particular time interval and/or in vivo location are quantitatively assessed by comparing the number of armed and labeled cells that were reinfused with the number of armed and labeled cells present in a sample at the particular time interval and/or in vivo location by phenotyping the cellular population using flow cytometry.

14. The method of claim 13, wherein blood samples and target tissue samples taken from a patient at a particular time interval and quantitatively assessed using flow cytometry, is indicative of in vivo homing progress of armed and labeled stem cells to target tissues.

15. A method of treating a patient suffering from cancer, comprising the steps of:
(a) isolating peripheral blood mononuclear cells from a patient suffering from cancer;
(b) activating of T cells by ex vivo stimulation with soluble anti-CD3 monoclonal antibody;
(c) arming of unactivated and/or activated T cells with bispecific antibodies capable of binding to the T cell receptor complex of a T cell, and to tumor-associated antigens on a tumor cell, under conditions wherein;
(i) bispecific antibody binds to said T cells, tumor cells, and Fc-receptor positive cells,
(ii) activation of said T cells by said antibody binding to the tumor target,
(iii) redirection of said T cells and Fc-receptor positive cells to said tumor cells,
(iv) destruction of said tumor cells by said activated and armed T cells; and,
(d) binding of a labeled secondary antibody specific for the Fc region of the bispecific antibody; or,
(e) directly labeling the bispecific antibody with a detectable marker; and,
(f) reinfusing the armed and labeled activated T cells into a patient.

16. The method of claim 15, wherein the bispecific antibody is comprised of two monoclonal antibodies.

17. The method of claim 15, wherein each of the specificities of the bispecific antibody are directed to a tumor antigen and the T cell receptor complex.

18. The method of claim 17, wherein the bispecific antibody is comprised of monoclonal antibodies directed to any tumor associated antigen.

19. The method of claim 17, wherein the anti T cell receptor monoclonal antibody component of the bispecific antibody is directed against CD3 of the T cell receptor complex.

20. The method of claim 16, wherein secondary antibody, binding to the Fc region of the bispecific antibody, is fluorescently labeled.

21. The method of claim 16, wherein the bispecific antibody is directly fluorescently labeled.

22. The method of claim 16, wherein the armed and labeled T cells are redirected to the tumor antigens.

23. The method of claims 16 through to 22, wherein samples are taken at different time intervals after reinfusion of the T cells to track the location of the armed and labeled T cells.

24. The method of claim 23, wherein the numbers of armed and labeled cells at a particular time interval and/or in vivo location are quantitatively assessed by comparing the number of armed and labeled cells that were reinfused with the number of armed and labeled cells present in a sample at the particular time interval and/or in vivo location by flow cytometry.

25. The method of claim 23, wherein blood samples and target tissue samples taken from a patient at a particular time interval and quantitatively assessed using flow cytometry, is indicative of in vivo homing progress of armed and labeled T cells to target tumors.

26. A method for tracking cells it vivo at any desired location, the method comprising:
isolating and purifying cells from a subject; and,
providing a chemically heteroconjugated bispecific antibody with a binding site specific for cellular antigen and a binding site specific for a target antigen in any location in a patient; and,
arming the isolated cells with the bispecific antibody under conditions wherein;
(i) the bispecific antibody binds to a specific antigen on the isolated cell; and,
(ii) the second antigenic binding site of the bispecific antibody is free to bind to a target antigen; and,
(iii) binding of a labeled antibody to the Fc region of the bispecific antibody; or,
(iv) fluorescently labeling the bispecific antibody thereby a secondary labeled antibody is not required; and,
(d) reinfusing the armed and labeled cells into a patient; and,
(e) tracking the armed and labeled cells by extracting samples from the patient at different time intervals; and,
(f) identifying the armed and labeled cells using flow cytometry cell sorting; and,
(g) identifying the armed and labeled cells by immunohistochemical staining or other methods to detect the primary antibody on the cells in various target tissues such as bone marrow, spleen, liver, pancreas, lungs, neural tissue, gastrointestinal track, heart, vascular endothelium.

27. The method of claim 26, wherein the isolated cells are bone marrow cells.

28. The method of claim 26, wherein the isolated cells are hematopoietic stem cells.

29. The method of claim 26, wherein the isolated cells are erythroid stem cells.

30. The method of claim 26, wherein the isolated cells are cells of the immune system.

31. The method of claims 26 Through 30, wherein the bispecific antibody binds to a specific ligand of the desired cell.

32. The method of claim 31, wherein the bispecific antibody is directed to a specific antigen on a tissue, organ, or cells.

33. The method of claim 31, wherein trafficking and homing of the armed and labeled cells is detectable by a fluorescent label using flow cytometry.

34. The method of claims 1, 15, 26 or 33, wherein the fluorescent label is selected from the group consisting of green, red, blue, green, cyan, and yellow.

35. The method of claim 34, wherein the isolated cells are transformed with nucleic acid molecules which encode for fluorescent proteins.

36. The method of claim 35, wherein the fluorescent protein is green fluorescent protein.

37. The method of claim 35, wherein the fluorescent protein is enhanced green fluorescent protein.

38. The method of claim 35, wherein the fluorescent protein is red fluorescent protein.

39. A composition comprising: isolated and purified cells, a vector encoding for a bispecific antibody and a fluorescent protein, wherein the isolated cells are transformed with the vector.

40. The composition of claim 39, wherein the isolated cell is a stem cell.

41. The composition of claim 40, wherein the vector is further comprised of oligonucleotides encoding complementary mRNA to specific target mRNA which codes for cell surface antigens.

42. The composition of claim 41, wherein the complementary mRNA inhibits the cell surface expression of cell surface antigens involved in autoimmune or inflammatory diseases.

43. The composition of claim 42, wherein the isolated cell expresses the bispecific antibody on its surface.

44. The composition of claim 43, wherein the isolated cell is targeted to a specific location in vivo.

45. The composition of claim 44, wherein the isolated cell differentiates into the mature cell of the targeted location and does not express the cell surface antigen involved in autoimmune disease or inflammatory disease.

46. The composition of claim 45, wherein the isolated cell is used to provide therapy to a patient suffering from or susceptible to autoimmune and/or inflammatory diseases.

47. The composition of claim 46, wherein the autoimmune disease is rheumatoid arthritis.

48. The composition of claim 46, wherein the autoimmune disease is diabetes.

49. The method of claims 1, 15, 26 or 33, wherein isolated and purified cells are used in functional assays.

50. The method of claims 1, 15, 26 or 33, the functional assays are determined by the cell type and desired cell property.

51. The method of claim 50, wherein the isolated and purified cell is a lymphocyte.

52. The method of claim 51, wherein the functional assay is a T cell assay.

53. The method of claim 51, wherein the functional assay is an ELlSA, RIA.

54. The method of claim 51, wherein the functional assay is a cytoline assay.
